(19) United States
(12) Patent Application Publication
(10) Pub. No.: US 2026/0191947 A1
(43) Pub. Date: Jul. 9, 2026

(54) AFFORDABLE UNIVERSAL FUSION UNIVERSAL VACCINE FOR FOOT AND MOUTH DISEASE INFECTIONS

(71) Applicant: Matthias Magoola, Kampala (UG)

(72) Inventor: Matthias Magoola, Kampala (UG)

(21) Appl. No.: 19/012,157

(22) Filed: Jan. 7, 2025

(51) Int. Cl. A61K 39/135; A61K 39/00

(52) U.S. Cl. CPC A61K 39/135; A61K 2039/53; A61K 2039/552; A61K 2039/55544; A61K 2039/6031; A61K 2039/6081

(57) ABSTRACT

The present invention relates to a pharmaceutical composition for inducing one or more immune responses capable of cross-protection against multiple strains or serotypes of a virus to generate protection with a single vaccination campaign. This universal vaccine comprises a composition presenting epitopes of antigens of all known sera of A, O, C, Asia, and the South African types of SAT-1, 2, and 3 capable of developing immunity against the Foot and Mouth Disease (FMD) in food-producing animals, such as cattle, sheep, goats, swine and other cloven-hoofed animals. The antigen vaccine is fused with serum proteins or short peptides to bind to serum proteins to enhance its half-life and with an adjuvant to enhance the activity of the vaccine when produced using recombinant technology. The RNA-based delivery of vaccines reduces the cost significantly, making this universal fusion vaccine affordable globally.

### SEQUENCE LISTING

The instant application contains a Sequence Listing, which has been submitted electronically in ASCII format and is hereby incorporated by reference in its entirety. Said ASCII text file, created on Jan. 11, 2025, is named ST26SequenceListing, and is 74 kb in size.

### SPECIFICATION

### Field of the Invention

The present invention relates generally to the field of vaccine compositions. In one embodiment, it is a universal fusion universal vaccine combining the epitopes of all known antigen-producing epitopes capable of inducing cross-protection against all known serotypes and strains of viruses responsible for foot and mouth disease (FMD) to create a complete universal antigen that, in another embodiment, is fused with a serum protein or to a small synthetic peptide that binds to a serum protein to extend the half-life of the antigen that is further connected to an adjuvant, when expressing the antigen using recombinant technology; in another embodiment, the present invention presents RNA technology, where the antigen is expressed through linear mRNA, circular RNA and both non-replicating and replicating to substantially reduce the cost of goods (COGS), bringing these vaccines within the affordable reach of the entire world.

### Background of the Invention

With the global population expanding at its current rate, it is crucial to ensure a safe food supply that is both secure and sustainable. The animal health industry is a key player in this challenge and strives to develop advanced solutions for a safe, secure, and sustainable food supply. Animal health products contribute to improving and maintaining the health and welfare of animals in terms of disease prevention, treatment, and control. One of the most important causes of animal diseases is infections by viruses. Some are contagious, can produce devastating economic losses, and impact public health.

Foot and Mouth Disease (FMD) is an acute systemic viral infection affecting food-producing animals, such as cattle, sheep, goats, swine, and other cloven-hoofed animals. Despite its very low mortality rate, the highly contagious nature of FMD makes it one of the most serious diseases of the livestock industry in terms of productivity losses and economic impact.

FMD is endemic in many parts of the world. The World Organization for Animal Health (OIE) periodically publishes disease distribution and outbreak world maps. The sanitary status granted by the OIE has a profound economic impact on countries with meat trade-dependent economies because of the market restrictions imposed in countries affected by the Foot and Mouth Disease Virus (FMDV). Other viruses that can also cause significant impacts on livestock productivity include, but not limited to, Bovine Rotavirus which is the causative agent of neonatal diarrhea in calves, Bovine Herpesviruses 1 and 5 (BoHV-1 or BHV-1 and BoHV-5 or BHV-5) which are the etiologic agents of Infectious Bovine Rhinotracheitis and Bovine Herpetic Encephalitis respectively, Bovine Parainfluenza Virus 3 (PI3 o BPIV-3) and Bovine Respiratory Syncytial Virus (BRSV) which are both associated with the bovine respiratory disease (BRD) complex, Bovine Viral Diarrhea Virus (BVDV) which, in addition to diarrhea, can cause immunosuppression, abortion, infertility and a fatal complication called mucosal disease and the Rabies Virus which is the pathogen causative of lethal encephalitis in both animals and human beings.

The FMDV is a non-lipid-enveloped virus featuring an icosahedral diameter of about 25-30 nm, containing a single-stranded RNA molecule of about 8500 nucleotides. The RNA molecule comprises a single open reading frame (ORF), encoding structural and non-structural proteins. Its proteome is divided into structural and non-structural proteins. The Foot-and-Mouth Disease (FMD) virus contains four structural proteins, VP1, VP2, VP3, and VP4, that form its capsid and are critical for its structure and infectivity. Among these, VP1 is the most extensively studied due to its significant roles in virus attachment to host cells, eliciting protective immunity, and determining serotype specificity. Its immunogenic properties make VP1 a primary target for developing peptide vaccines to provide specific and cross-serotype protection. In addition to the structural proteins, the virus has a group of non-structural proteins, including 2A, 2B, 2C, 3A, 3B, 3C, and 3D, which are essential for viral replication and intracellular functions. These non-structural proteins are instrumental in diagnostic assays designed to differentiate infected animals from vaccinated ones, a strategy known as DIVA (Differentiation of Infected and Vaccinated Animals). Vaccinated animals typically produce antibodies against structural proteins, while infected animals generate antibodies against structural and non-structural proteins. This distinction is crucial for controlling and eradicating FMD through targeted vaccination and diagnostic efforts.

Different serotypes of FMDV have been described, and each serotype is further divided into multiple strains. These serotypes include A, O, C, Asia, and the South African types of SAT-1, 2, and 3, with A, O, and Asia being the most common.

Despite continuous efforts to develop alternative vaccines against FMD that would not require propagation of the pathogen on a large scale, current vaccines are based on inactivated whole virus concentrated and purified to reach a critical antigen mass capable of generating a protective immune response. These vaccines are manufactured in Biosafety level 4 OIE (BSL4 OIE) facilities. It is estimated that between 2.5 and 3 billion doses are produced annually worldwide.

The global production of Foot-and-Mouth Disease (FMD) vaccines is substantial, reflecting the ongoing efforts to control this highly contagious livestock disease. While precise annual production figures are not readily available, the global FMD vaccine market was valued at approximately $2.2 billion in 2023 and is projected to reach $4.2 billion by 2033, growing at a compound annual growth rate (CAGR) of 6.5% from 2024 to 2033. This growth is driven by increased awareness of FMD's economic impact and the benefits of vaccination (https://www.alliedmarketresearch.com/foot-and-mouth-disease-vaccines-market-A324424?)

The market valuation of foot-and-mouth disease (FMD) vaccines highlights the production of hundreds of millions of doses annually to meet global demand, primarily due to their relatively low cost and widespread need. FMD vaccines are priced between $0.50 and $3 per dose, which remains substantial for resource-constrained settings. Additionally, decision-support tools like VADEMOS have been developed to estimate current and future vaccine dose demand at national, regional, and global levels, aiming to bridge the gap between FMD vaccine demand and production in endemic countries (https://www.fao.org/eufmd/tools/vademos-model-for-vaccine-demand/en/?)

Additionally, the CAPEX to establish production of these vaccines is prohibitive for regions where these vaccines are direly needed, such as Africa. Therefore, there is a need to develop a cost-effective solution. The present invention presents the most cost-effective option expression of a universal FMD vaccine in a self-replicating circular RNA that reduces the CAPEX as no biosafety issues are involved and produces a million times the number of molecules for each molecule of the RNA, making it possible to manufacture these vaccines in small facilities at almost negligible cost.

Peptide vaccines are a safer and more economical technology than traditional vaccines. The disadvantage of this technology is its poor immunogenicity. Several experiments have been performed with this vaccine to test its ability to protect animals. The published results showed that the protection reached with the peptide vaccine was lower than 50% in all the challenges tested in the field. In contrast with these results, inactivated virus vaccines (positive control) commonly reach from 90 to 100% of protection. For this reason, the vaccines routinely used as part of eradication programs and in emergencies are based on inactivated viruses.

Another example of a peptide vaccine involves the use of dendrimeric peptides. These peptides comprise a core of lysine residues, two or more branches of amino acids, and T and B epitopes in their N- and C-terminal, respectively. Initially, these dendrimeric peptides were used as antimicrobial peptides, but currently, they are used as multi-antigenic peptides for animal vaccination. In one patent application (EP2647390A1), the inventors showed that they had developed a dendrimeric peptide that could elicit a homologous immune response higher than the linear peptide. The disadvantage of these dendrimer peptides is the necessity of formulating the vaccine with a high quantity of these peptides to confer solid protection against FMDV. This is genuinely problematic because large-scale manufacture of such vaccines is economically non-viable.

Different serotypes of FMDV are distributed around the world. Some regions have more than one serotype and several strains, which complicate the sanitary situation and even hinder the eradication of this disease. Due to the great importance of FMD in economic losses, it is crucial to have a vaccine that can provide cross-protection against more than one serotype and/or strain of FMDV. Thus, animals in a specific region could be protected extensively with fewer vaccination campaigns.

A significant difficulty in formulating vaccines for FMDV is the remarkable antigenic diversity that this virus presents, particularly the VP1 protein, which displays a high degree of genetic variation. A high degree of genetic variation accounts for the lack of cross-protection among serotypes. When animals are vaccinated against or recovered from a virus of one serotype, they are still susceptible to viruses from the other six serotypes. Moreover, a high degree of antigenic variation within a serotype may cause a vaccine protective against one strain to become ineffective against another strain within that same serotype.

There is a lack of protective response afforded by vaccines based on only one specific strain when used against another. There is no reactivity between one strain and the monoclonal antibody of the other. To overcome the lack of cross-protection problem and given the wide range of different peptides that currently exist, tailor-made peptide vaccines could help resolve this issue due to their ability to change the vaccine target easily. Although peptide vaccines seem to be a good strategy for cross-protection, this vaccine is weekly immunogenic and unviable economically. Moreover, they do not elicit a strong cell-mediated immune response, which is the cornerstone of achieving total immunogenic protection. Indeed, FMDV-specific cell-mediated immune response depends on the integrity and stability of the virus capsid antigen; therefore, peptide vaccines cannot trigger a solid cell-mediated reaction. In addition, it has been demonstrated that cell-mediated immune response is crucial for cross-reactive protection against heterologous strains. Thus, adding an inactivated FMDV to the vaccine formulation is highly recommended to trigger a stronger humoral and cell-mediated immune response.

For the issues identified above, it is proposed to combine an inactivated FMD whole virus antigen with other vaccine technologies to take advantage of the abilities of the inactivated antigen to induce strong cellular immunological response and to use the novel vaccine technologies to achieve broad cross-protection and specificity through the triggering of strong and broad antibody responses. This novel universal vaccine against FMD represents a great tool in the fight against the FMDV pandemics worldwide. It will solve an unmet market and technical need to afford total protection against all serotypes and different strains of FMDV. Among the novel technologies, peptide antigens are excellent candidates for combination with inactivated antigens because of the multi-target ability of peptide vaccines.

The duration of activity of cattle peptide vaccines, such as those used to protect against foot-and-mouth disease (FMD), is often limited due to the rapid clearance of peptides from the bloodstream through renal filtration and enzymatic degradation. To enhance their efficacy and reduce the frequency of booster doses, peptide vaccines can be conjugated with carriers such as albumin, transferrin, or the Fc domain of immunoglobulin. These conjugates extend the half-life of the peptide vaccine and improve its immunogenicity. Albumin conjugates leverage the natural recycling mechanism of albumin through its interaction with the neonatal Fc receptor (FcRn), significantly prolonging systemic circulation from hours to days. This can be achieved through direct fusion of peptides with albumin or by using albumin-binding peptides that attach non-covalently in vivo. Similarly, transferrin conjugates enhance delivery to antigen-presenting cells (APCs) by binding transferrin receptors (TfR) on immune cells, facilitating receptor-mediated endocytosis and efficient antigen presentation to T cells. In another strategy, Fc conjugates exploit interactions with Fc receptors on immune cells and FcRn recycling to extend the half-life of peptides and amplify immune responses via Fcγ receptor-mediated pathways. Fc fusion proteins can present peptides in multivalent formats, improving their immunogenic potential. Examples of such conjugates include peptide-albumin fusions for prolonged circulation, peptide-transferrin constructs for targeted lymphoid tissue delivery, and Fc-conjugated peptides for enhanced immune cell activation.

Additionally, these conjugates can be paired with adjuvants such as TLR agonists (e.g., CpG ODNs or Poly(I:C)), saponin-based adjuvants like QS-21, or oil-in-water emulsions such as MF59 to boost both cellular and humoral immune responses further. Challenges, however, include ensuring the stability of the conjugates during production and storage, avoiding unwanted immune responses to the carrier, and optimizing scalability for large-scale vaccine production. By using these conjugation strategies, cattle peptide vaccines can achieve extended duration of activity, improved immune responses, and reduced frequency of administration, making them more effective and practical for widespread livestock immunization.

Table 1 lists cattle plasma proteins that make a good target for increasing the potency of the FMD.

TABLE 1
Cattle plasma proteins

| | | Molecular | Approximate | |
|---|---|---|---|---|
| Protein | UniProt ID | Weight (kDa) | Half-Life | |
| Albumin | P02769 | ~66.5 | 19-21 | days |
| (BSA) | | | | |
| IgG2 (Fc | F1MHH9 | ~150 (monomeric) | 14-16 | days |
| gamma) | | | | |
| IgG1 | Q3SZR3 | ~150 (monomeric) | 7-8 | days |
| Transferrin | Q29443 | ~78.4 | 8-10 days | |
| | | | (estimated) | |
| Complement | Q28085 | ~158 | 5-7 days | |
| Factor H | | | (human analog) | |
| Fibrinogen | P02672 (alpha) | ~340 (hexamer) | 4 | days |
| | P02675 (beta) | | | |
| | P12799 | | | |
| | (gamma) | | | |
| Thyroglobulin | P01267 | ~660 (homodimer) | 2-3 | days |
| Factor IX | P00741 | ~55.4 | 18-24 hours | |
| | | | (human analog) | |
| C-Reactive | C4T8B4 | ~23 (monomeric) | 18 hours | |
| Protein | | ~115 (pentameric) | (human analog) | |
| Retinol-Binding | G1K122 | ~21 | 12 | hours |
| Protein (RBP) | | | | |
| Transcription | A0AAF6 | ~50 | 5 | hours |
| Factor VIIa | | | | |
| G-CSF | P35833 | ~19 | 3.5-4 | hours |
| Interferon-Alpha | P05007 | ~19 | 2-4 | hours |
| (IFN-α) | | | | |

The Fc region of bovine immunoglobulin G (IgG) is a horseshoe-shaped homodimer that interacts with other parts of the immune system to promote the removal of antigens. The Fc region mediates the effector functions of IgG subclasses, which include: phagocytosis, antibody-dependent cellular cytotoxicity, release of inflammatory mediators, immune complex clearance, and regulation of antibody production.

For the first time, the present invention introduces a platform for obtaining a wide range of vaccines that combine polynucleotides encoding viral peptides or recombinant viral peptides containing adjuvants, emulsifiers, molecular adjuvants, and carrier systems. It is proposed that the present invention could develop a universal vaccine to protect an animal against one or more viral diseases, particularly the FMD.

### SUMMARY OF THE INVENTION

In one embodiment, the present invention provides a detailed procedure for formulating a universal vaccine capable of protecting against various serotypes and/or strains of FMD viruses.

In one embodiment, the formulations are composed of polynucleotides encoding viral peptides, polypeptides, or proteins in different types of plasmids; viral peptides or recombinant viral peptides, along with a carrier or as molecular adjuvant fused to peptides, polypeptides and/or proteins derived from viruses; adjuvants; emulsifiers, molecular adjuvants, and carrier systems.

In one embodiment, the present invention discloses a vaccine formulation capable of inducing cross-protection against different serotypes or strains of FMD viruses by (a) polynucleotides encoding peptides, polypeptides or proteins of the virus; (b) recombinant peptides, polypeptides or proteins of the virus; (c) peptides, polypeptides or proteins fused with serum proteins and (d) adjuvants to enhance the potency of the FMD vaccine.

In one embodiment, the pharmaceutical combination comprising one or more vaccine formulations of the present invention can ensure high protection against FMDV by induction of cell-mediated and humoral components of the immunological response.

In one embodiment, the pharmaceutical combination of the present invention could be administered to cloven-hoofed hosts, such as cattle, sheep, goats, or swine, to induce an immune response against FMD.

In one embodiment, the pharmaceutical combination comprising one or more vaccine formulations of the present invention has the advantage of generating immunogenic cross-protection with fewer vaccination campaigns.

Extending the half-life of foot-and-mouth disease (FMD) antigens by conjugating them with serum proteins in cattle is a promising approach to improve vaccine efficacy and reduce dosing frequency. Below is an analysis of serum protein conjugation strategies specifically tailored for FMD vaccines: FcRn binds to the Fc region of IgG or albumin at acidic pH in endosomes, preventing degradation and recycling these proteins back into circulation, Exploiting Serum Protein Affinity, PEGylation, Targeting Other Serum Proteins such as Haptoglobin, Transferrin.

In one embodiment, the present invention provides a pharmaceutical combination comprising a virus peptide combined with an adjuvant or adjuvant, as shown in Table 2.

TABLE 2
The choice of adjuvants for the FMD vaccine depends
on the adjuvant's introduction method.

| | Recombinant | In Vitro Chemical | mRNA-Based |
|---|---|---|---|
| Adjuvant | Technology | Conjugation | Delivery |
| Alpha-Gal-Based | Include alpha-gal | Chemically | Encode alpha-gal |
| Adjuvants | epitopes in the | conjugate antigens | epitopes in mRNA |
| | recombinant antigen | to alpha-gal- | for co-expression |
| | to enhance | containing | with the antigen. |
| | immunogenicity. | molecules for | |
| | | enhanced | |
| | | recognition by | |
| | | APCs. | |
| Alpha-Galactosylceramide | Genetically modify | Conjugate to | Co-encapsulate α- |
| (α-GalCer) | antigens for | antigens via lipid | GalCer with mRNA |
| | conjugation to α- | anchors or | in lipid |
| | GalCer derivatives. | carbohydrate- | nanoparticles to |
| | | reactive | activate NKT cells. |
| | | chemistries. | |
| Beta-Glucans | Not suitable for | Conjugate to | Combine with |
| | genetic fusion.; Co- | antigens via | mRNA |
| | administer with | carbohydrate- | formulations in |
| | recombinant | reactive | nanoparticles for |
| | proteins to enhance | chemistries.; | co-delivery and |
| | APC activation. | Combine with | enhanced APC |
| | | nanoparticles for | activation. |
| | | adsorption. | |
| C3d (Complement | Genetically fuse to | Conjugate to | Encode as part of |
| Component) | the antigen to | antigens via amine | the mRNA |
| | enhance B-cell | or thiol-reactive | sequence |
| | activation and | chemistries. | downstream of the |
| | complement | | antigen to improve |
| | fixation. | | B-cell activation. |
| CAF01 (Cationic | It is not fused | Mix antigen with | Use as an |
| Liposomes) | genetically; it is | CAF01 liposomes | alternative to LNPs |
| | used as a delivery | for adsorption or | for encapsulating |
| | vehicle for | encapsulation.; No | mRNA.; Combine |
| | recombinant | covalent | with mRNA-loaded |
| | proteins. | conjugation is | liposomes for co- |
| | | required. | delivery. |
| Calcium Phosphate | Adsorb | Co-encapsulate or | Calcium phosphate |
| Nanoparticles | recombinant | adsorb antigens | nanoparticles can |
| | proteins onto | onto calcium | be used as carriers |
| | calcium phosphate | phosphate | for mRNA to |
| | particles for | nanoparticles. | enhance |
| | delivery; no genetic | | immunogenicity |
| | fusion is required. | | and cellular uptake. |
| Chitosan (for mucosal | Combine | Adsorb antigen to | Use chitosan |
| vaccines) | recombinant protein | chitosan | nanoparticles to |
| | with chitosan | nanoparticles for | encapsulate mRNA |
| | particles for | mucosal | for mucosal |
| | mucosal delivery. | administration.; No | delivery. |
| | | direct conjugation. | |
| Chitosan Microparticles | Combine with | Encapsulate | Use chitosan |
| | recombinant | antigen in chitosan | microparticles to |
| | proteins for mucosal | particles or adsorb | encapsulate mRNA |
| | delivery; no genetic | it onto the surface. | for mucosal |
| | fusion is needed. | | delivery. |
| Cholera Toxin B Subunit | Genetically fuse | Conjugate CTB to | Encode CTB as |
| (CTB) | antigens to CTB for | antigens via amine- | part of the mRNA |
| | enhanced mucosal | reactive | sequence to |
| | immune responses. | chemistries; co- | promote mucosal |
| | | administered for | immunity, |
| | | mucosal vaccines. | especially for |
| | | | intranasal or oral |
| | | | vaccines. |
| CpG ODN (TLR9 agonist) | Not suitable for | Adsorb onto | Co-encapsulate |
| | genetic fusion.; Co- | nanoparticles or | with mRNA in |
| | administer with | emulsions.; | LNPs.; Separate |
| | recombinant antigen | Conjugate to | delivery in |
| | in formulations. | antigens via linker | combination with |
| | | arms. | mRNA vaccine. |
| Cyclic Dinucleotides (e.g., | It is unsuitable for | Formulate with | Co-encapsulate |
| c-di-GMP, c-di-AMP) | genetic fusion and | antigens in | cyclic dinucleotides |
| | co-administered | nanoparticles or | with mRNA in |
| | with recombinant | liposomes; no | LNPs to enhance |
| | proteins. | chemical | activation of |
| | | conjugation is | STING pathways in |
| | | needed. | APCs. |
| Cytosine-Phosphate- | It is unsuitable for | Mix or adsorb CpG | Co-encapsulate |
| Guanine Dinucleotides | genetic fusion and | onto nanoparticles | CpG with mRNA in |
| (CpG ODNs) | co-administered | with antigens; no | LNPs for enhanced |
| | with recombinant | chemical | dendritic cell |
| | antigens. | conjugation is | activation and Th1 |
| | | required. | responses. |
| Defensins (Other than | Genetically fuse to | Conjugate to | Encode defensins |
| hBD-3) | antigens for | antigens via | along with antigens |
| | enhanced innate | reactive residues | in the same mRNA |
| | immune activation. | (e.g., lysine or | sequence for co- |
| | | cysteine). | expression. |
| Dendritic Cell-Targeting | Fuse antigen | Conjugate antigens | Encode scFv |
| Antibodies | genetically to | to antibodies via | targeting sequences |
| | single-chain | linker chemistries | along with the |
| | antibodies (scFvs) | (e.g., NHS-ester or | antigen in the |
| | targeting dendritic | maleimide cross- | mRNA sequence. |
| | cell receptors (e.g., | linkers). | |
| | DEC-205). | | |
| Flagellin (TLR5 agonist) | Genetically fuse to | Not suitable for | Encode flagellin as |
| | the antigen for | chemical | part of the mRNA |
| | recombinant | conjugation. | sequence, either |
| | expression.; | | upstream or |
| | Maintain TLR5 | | downstream of the |
| | activation domain in | | antigen. |
| | fusion design. | | |
| Flagellin Variants (e.g., | Genetically modify | It is unsuitable for | Encode FliC |
| FliC) | antigens to fuse | chemical | variants in mRNA |
| | with flagellin | conjugation; it is | constructs for |
| | domains to enhance | part of formulations | synergistic |
| | TLR5 activation. | for innate immune | activation of TLR5 |
| | | activation. | and antigen |
| | | | presentation. |
| Flt3 Ligand | Genetically fuse to | Conjugate Flt3 | Encode as part of |
| | the antigen to | ligand to antigens | the mRNA |
| | enhance dendritic | via bifunctional | construct to |
| | cell recruitment and | linkers. | promote dendritic |
| | activation. | | cell expansion and |
| | | | antigen |
| | | | presentation. |
| GM-CSF (Cytokine) | Fuse with antigen | Conjugate to | Encode GM-CSF |
| | as part of a | antigens using | as a separate mRNA |
| | recombinant protein | bifunctional linkers, | strand or in the |
| | to recruit APCs. | such as maleimide | same construct as |
| | | derivatives.; | the antigen.; Co- |
| | | Separate co- | encapsulate in |
| | | administration. | LNPs. |
| Heat Shock Protein 70 | Genetically fuse | Conjugate HSP70 | Encode HSP70 and |
| (HSP70) | antigens to HSP70 | to antigens via | antigen together in |
| | to enhance antigen | chemical cross- | the same mRNA |
| | presentation and | linkers targeting | sequence for |
| | cross-presentation | amines or thiols. | intracellular co- |
| | by APCs. | | expression. |
| Heat-Inactivated Bacteria | Not suitable for | Combine antigens | Combine mRNA |
| | genetic fusion.; Co- | with inactivated | formulations with |
| | administer heat- | bacteria; no | heat-inactivated |
| | killed bacterial | chemical | bacteria to mimic |
| | adjuvants with | conjugation is | pathogen-associated |
| | recombinant | required. | molecular patterns |
| | proteins. | | (PAMPs). |
| Human β-Defensin 3 | Genetically fuse to | Covalently link via | Encode as part of |
| (hBD-3) | antigen with | lysine (amine- | the same mRNA |
| | flexible or cleavable | reactive) or cysteine | sequence with a |
| | linkers.; Express as | (thiol-reactive) | linker.; Co-deliver |
| | a single polypeptide | residues.; Use | as a separate mRNA |
| | in a host system. | EDC/NHS or | encapsulated in |
| | | maleimide | LNPs. |
| | | chemistries. | |
| Hyaluronic Acid | Use as a delivery | Conjugate antigens | Combine with |
| Derivatives | vehicle for | to hyaluronic acid | mRNA-loaded |
| | recombinant | derivatives for | nanoparticles for |
| | proteins; no genetic | localized delivery. | enhanced delivery |
| | fusion is required. | | to dendritic cells or |
| | | | mucosal tissues. |
| IL-12 (Cytokine) | Fuse to antigen in a | Mix or co- | Encode IL-12 as a |
| | recombinant | administer.; | separate mRNA |
| | construct, though | Chemical | strand or co- |
| | functionality may | conjugation is | expressed with |
| | be affected. | uncommon due to | antigen.; Co- |
| | | cytokine sensitivity. | encapsulate in |
| | | | LNPs. |
| Imiquimod (TLR7 | Not suitable for | Conjugate to | Co-encapsulate |
| agonist) | genetic fusion.; | lipophilic anchors | with mRNA in lipid |
| | Used topically or | for delivery | nanoparticles for |
| | co-delivered with | systems.; Mix with | synergistic immune |
| | antigen. | nanoparticles for | activation. |
| | | adsorption. | |
| Immunostimulatory | Co-administer with | Adsorb antigen | Combine |
| Complex (ISCOMATRIX) | recombinant | onto | ISCOMATRIX with |
| | proteins.; Cannot be | ISCOMATRIX | mRNA |
| | genetically fused. | particles; no direct | formulations to |
| | | chemical | improve antigen |
| | | conjugation is | delivery and APC |
| | | required. | activation. |
| Interferon-α (IFN-α) | Fuse with antigen | Co-administer with | Encode as a |
| | in recombinant | antigens in | separate mRNA |
| | constructs, though | formulations.; | strand for co- |
| | folding issues may | Conjugation | delivery in LNPs.; |
| | arise. | uncommon. | Co-express with |
| | | | antigen in the same |
| | | | mRNA sequence. |
| ISCOMs (Immune- | Use ISCOMs as | Mix ISCOMs with | Co-deliver mRNA |
| Stimulating Complexes) | carriers for | antigens for | with ISCOMs in |
| | recombinant | adsorption; no | nanoparticle |
| | antigens; no genetic | direct chemical | formulations. |
| | fusion. | conjugation is | |
| | | required. | |
| Leukotoxin Derivatives | Fuse antigen to | Chemically modify | Encode leukotoxin |
| | leukotoxin mutants | leukotoxins for | domains along with |
| | to enhance immune | antigen conjugation | antigens in mRNA |
| | activation; | via bifunctional | to increase immune |
| | genetically | linkers. | activation. |
| | engineered. | | |
| MF59 (Oil-in-Water | Not suitable for | Physical | Combine with |
| Emulsion) | genetic fusion.; Co- | emulsification with | mRNA-LNP |
| | administer with | antigen; no covalent | formulations or co- |
| | recombinant | conjugation. | deliver as a separate |
| | antigens. | | emulsion. |
| Microneedle Patches | Deliver | Adsorb antigens | Encapsulate |
| | recombinant | onto microneedles | mRNA within |
| | proteins using | or encapsulate | microneedles for |
| | microneedle | within dissolvable | transdermal |
| | systems; no genetic | microneedles. | delivery. |
| | fusion is required. | | |
| Monophosphoryl Lipid A | Link to antigens | Covalently | Incorporate in |
| (MPL) | through lipid | conjugate to | LNPs along with |
| | moieties for | antigens via lysine | mRNA.; Co-deliver |
| | recombinant | residues or | as part of the lipid |
| | formulations. | lipophilic anchors. | formulation. |
| Muramyl Dipeptide | Not suitable for | Adsorb onto | Co-encapsulate |
| (MDP) | genetic fusion.; Co- | nanoparticles or | with mRNA in |
| | delivered with | emulsify with | LNPs for enhanced |
| | recombinant | antigens; no | immune activation |
| | proteins. | chemical | via NOD2 |
| | | conjugation is | pathways. |
| | | required. | |
| Nanodiamonds | Adsorb | Conjugate antigens | Combine |
| | recombinant | to nanodiamonds | nanodiamonds with |
| | proteins onto | via covalent or | mRNA for |
| | nanodiamonds; no | electrostatic | enhanced delivery |
| | genetic fusion is | interactions. | and cellular uptake. |
| | needed. | | |
| PADRE (Pan HLA-DR | Genetically include | It is unsuitable for | Encode as part of |
| Epitope) | PADRE sequence | chemical | the mRNA |
| | within the antigen | conjugation; it is | sequence to ensure |
| | construct to enhance | used as a peptide | the PADRE peptide |
| | CD4+ T cell | for co- | is co-expressed |
| | responses. | administration. | with the antigen. |
| Peptidoglycan Fragments | It is unsuitable for | Adsorb antigens | Combine |
| | genetic fusion and | onto peptidoglycan | peptidoglycan |
| | co-administered | fragments for | fragments with |
| | with recombinant | immune activation; | mRNA |
| | proteins. | no chemical | formulations to |
| | | conjugation is | mimic pathogen- |
| | | needed. | associated |
| | | | molecular patterns |
| | | | (PAMPs). |
| Plant-derived saponins | Co-administer with | Formulate with | Combine with |
| (e.g., Ginseng Saponins) | recombinant | antigens in | mRNA-loaded |
| | proteins.; Not | emulsions or | LNPs for co- |
| | suitable for genetic | liposomes; no | delivery in vaccine |
| | fusion. | chemical | formulations. |
| | | conjugation is | |
| | | required. | |
| PLGA Nanoparticles | Encapsulate | Adsorb antigen to | Use PLGA |
| | recombinant protein | nanoparticles.; No | nanoparticles to |
| | along with PLGA | direct covalent | encapsulate mRNA |
| | nanoparticles. | conjugation is | and stabilize |
| | | required. | adjuvant-antigen |
| | | | interactions. |
| Poly(I:C) (TLR3 agonist) | Not suitable for | Mix with the | Co-encapsulate |
| | genetic fusion.; Co- | antigen in | with mRNA in |
| | administer with | emulsions.; No | LNPs for |
| | recombinant antigen | covalent | synergistic innate |
| | formulations. | conjugation is | immune activation. |
| | | required. | |
| Polymer-based adjuvants | Co-deliver with | Adsorb or | Use PEI-based |
| (e.g., Polyethyleneimine, | recombinant | encapsulate | nanoparticles to |
| PEI) | proteins; no genetic | antigens within | encapsulate mRNA, |
| | fusion required. | PEI-based | enhancing cellular |
| | | nanoparticles. | uptake and immune |
| | | | activation. |
| Polyphosphazene (e.g., | Combine with | Mix antigens with | Co-encapsulate |
| PCEP) | recombinant protein | PCEP for | with mRNA in |
| | formulations; no | adsorption or co- | nanoparticles or |
| | genetic fusion is | delivery; no | mix for co- |
| | needed. | chemical | administration. |
| | | conjugation. | |
| QS-21 (Saponin-based) | Combine with | Mix antigen with | Co-encapsulate |
| | recombinant protein | QS-21 in liposomal | with mRNA in |
| | in adjuvant systems | formulations.; | liposomes or |
| | like AS01 | Adsorption or | nanoparticles.; |
| | (liposomal | physical | Separate delivery |
| | formulations). | conjugation. | with mRNA |
| | | | vaccines. |
| R848 (TLR7/8 agonist) | Not suitable for | Adsorb onto | Co-encapsulate |
| | genetic fusion.; Co- | nanoparticles or | with mRNA in |
| | administer with | conjugate to | LNPs for robust |
| | recombinant | lipophilic agents for | TLR7/8-mediated |
| | proteins. | enhanced delivery. | immune activation. |
| Resiquimod (R848, | It is unsuitable for | Adsorb or co- | Co-encapsulate |
| TLR7/8 agonist) | genetic fusion and | encapsulate R848 | R848 with mRNA |
| | co-delivered with | with nanoparticles | in LNPs for robust |
| | recombinant | for chemical | Th1-biased immune |
| | proteins. | stability and | responses. |
| | | immune activation. | |
| Retinoic Acid (RA) | Not suitable for | Conjugate to lipid | Co-encapsulate |
| | genetic fusion.; Co- | carriers for | retinoic acid with |
| | administer with | nanoparticle | mRNA in |
| | recombinant | formulations; no | nanoparticles to |
| | proteins to enhance | direct antigen | promote mucosal |
| | mucosal immunity. | conjugation is | immune responses. |
| | | required. | |
| Saponin-Based | Co-administer with | Mix antigens with | Co-encapsulate |
| Derivatives (e.g., Quil-A) | recombinant | Quil-A in emulsions | Quil-A with mRNA |
| | proteins in | or liposomes; no | in LNPs to enhance |
| | formulations like | chemical | immune responses. |
| | AS01. | conjugation is | |
| | | needed. | |
| Squalene-Based | Co-administer with | Mix antigen and | Combine with |
| Emulsions | recombinant | squalene emulsions | mRNA-LNP |
| | proteins; no genetic | (e.g., MF59 or | formulations or as |
| | fusion is needed. | AS03); no direct | separate adjuvant |
| | | conjugation. | delivery for |
| | | | systemic or |
| | | | mucosal immunity. |
| STING Agonists (e.g., | Not suitable for | Mix antigens with | Co-encapsulate |
| cGAMP) | genetic fusion.; Co- | STING agonists in | cGAMP with |
| | administer with | nanoparticles or | mRNA in lipid |
| | recombinant antigen | emulsions; no direct | nanoparticles to |
| | formulations. | conjugation is | enhance APC |
| | | required. | activation and |
| | | | cross-presentation. |
| Synthetic Nanoadjuvants | Not suitable for | Functionalize | Encapsulate |
| (e.g., Toll-Like Receptor | genetic fusion.; Co- | nanoparticles with | mRNA and |
| Agonist-Conjugated | deliver with | TLR agonists and | adjuvant- |
| Nanoparticles) | recombinant | adsorb antigens | functionalized |
| | proteins. | onto their surfaces. | nanoparticles for |
| | | | simultaneous |
| | | | antigen delivery |
| | | | and immune |
| | | | activation. |
| Synthetic RNA Sensors | Not suitable for | Co-formulate with | Co-encapsulate |
| (e.g., RIG-I Agonists) | genetic fusion.; Co- | nanoparticles for | RIG-I agonists with |
| | deliver with | antigen delivery; no | mRNA in LNPs to |
| | recombinant | direct conjugation | synergize with |
| | proteins. | required. | mRNA's innate |
| | | | immune activation. |
| Tetanus Toxin Fragment C | Fuse as a carrier | Conjugate to | Encode TTFc |
| (TTFc) | protein to improve | antigens via | alongside the |
| | immunogenicity of | chemical linkers | antigen in an |
| | poorly | (e.g., thiol-reactive | mRNA construct to |
| | immunogenic | agents). | enhance antigen |
| | antigens. | | processing. |
| TLR Ligand Mixtures | Combine multiple | Conjugate antigens | Encode |
| | TLR ligands | with multiple TLR | combinations (e.g., |
| | genetically fused to | ligands via | TLR3 + TLR9 |
| | the antigen to | chemical linkers or | agonists) in |
| | mimic pathogen- | nanoparticle | separate mRNA |
| | associated | delivery systems. | constructs for |
| | molecular patterns | | nanoparticle co- |
| | (PAMPs). | | delivery. |
| TLR2 Agonists (e.g., | Not suitable for | Adsorb antigens to | Co-encapsulate |
| Pam3CSK4) | genetic fusion.; Co- | nanoparticles | Pam3CSK4 with |
| | deliver with | containing TLR2 | mRNA in lipid |
| | recombinant | agonists; no | nanoparticles for |
| | proteins. | chemical | TLR2-mediated |
| | | conjugation is | immune activation. |
| | | needed. | |
| TLR4 Agonists (e.g., | It is unsuitable for | Mix with antigens | Co-encapsulate |
| GLA-SE) | genetic fusion and | in squalene | TLR4 agonists with |
| | co-administered | emulsions or | mRNA in LNPs to |
| | with recombinant | nanoparticles; no | promote innate and |
| | proteins in | direct conjugation | adaptive immune |
| | formulations. | is needed. | responses. |
| Toll-Like Receptor 3 | Co-administer with | Adsorb or | Co-encapsulate |
| Agonists (e.g., | recombinant | encapsulate | Poly(I:C) with |
| Polyinosinic:polycytidylic | proteins for | Poly(I:C) with | mRNA in LNPs to |
| acid, Poly(I:C)) | enhanced innate | nanoparticle | strengthen Th1- |
| | immune activation. | antigens; no | biased responses. |
| | | chemical | |
| | | conjugation is | |
| | | required. | |
| Trehalose Dibehenate | Fuse to antigens via | Adsorb antigens | Co-encapsulate in |
| (TDB) | recombinant | onto trehalose lipids | lipid nanoparticles |
| | expression for | or conjugate via | with mRNA for |
| | particulate vaccines. | lipid-modifying | enhanced delivery |
| | | agents. | and immune |
| | | | activation. |
| Trehalose-6,6-dimycolate | Co-deliver with | Adsorb or mix with | Co-encapsulate |
| (TDM) | recombinant | antigens in | TDM with mRNA |
| | proteins; not | nanoparticles or | in nanoparticles to |
| | suitable for genetic | emulsions; no direct | activate |
| | fusion. | conjugation. | macrophages and |
| | | | dendritic cells. |
| Uric Acid Crystals | It is unsuitable for | Co-administer with | Combine with |
| | genetic fusion; it is | antigens; no direct | mRNA in |
| | used to mimic | conjugation is | formulations for |
| | damage-associated | possible. | enhanced innate |
| | molecular patterns | | immunity |
| | (DAMPs). | | activation. |
| Virosomes (Lipid Bilayer | Fuse recombinant | Adsorb antigens | Use virosomes to |
| Vesicles with Viral | antigens to viral | onto virosomes or | deliver mRNA for |
| Components) | components | encapsulate within | simultaneous |
| | integrated into | lipid bilayers. | immune activation |
| | virosomes. | | and antigen |
| | | | expression. |
| Zymosan (TLR2/6 | It is unsuitable for | Adsorb antigens to | Combine zymosan |
| agonist) | genetic fusion; it is | zymosan particles; | with mRNA in |
| | used for innate | no direct | nanoparticle |
| | immune activation | conjugation is | formulations for |
| | via PRRs. | required. | enhanced innate |
| | | | immune responses. |

A short peptide that binds to albumin in cattle can be derived from well-characterized albumin-binding domains (ABDs) or albumin-binding peptides (ABPs). These peptides are engineered or naturally derived sequences designed to bind serum albumin with high affinity. While specific peptides optimized for bovine albumin are less common, strategies used for human or rodent albumin-binding peptides can be adapted for bovine albumin with some optimization. ABD-Derived Peptides derived from the albumin-binding domain of bacterial proteins (e.g., from Streptococcal Protein G), typically 13-46 amino acids in length such as DDDDKNQTPGNARILQTMKGL; synthetic Albumin-Binding Peptides are short peptides (5-12 amino acids) designed to bind albumin specifically such as DAHK or its derivatives (minimal functional motif derived from albumin itself).

The peptide sequence DDDDKNQTPGNARILQTMKGL is a synthetic peptide with potential utility for binding to albumin or other serum proteins, likely derived from studies focused on extending the pharmacokinetics of therapeutic agents or improving their delivery. The sequence KQNQQSSSEQDYSKKKK is a synthetic peptide that has been studied for its potential binding properties to various biological targets, including albumin and other serum proteins. However, to assess its relevance or utility in binding specific bovine targets like albumin, transferrin, or Fc regions.

DICLPRWGCLW is a 12-amino acid peptide known for its ability to bind albumin and potentially other serum proteins. Its sequence includes a combination of hydrophobic, polar, and charged residues, which contribute to its high-affinity binding properties.

DAHK (Asp-Ala-His-Lys) is a well-characterized peptide known to bind albumin across various species by targeting conserved binding sites. Due to the structural homology of albumin across species, including bovine serum albumin (BSA), DAHK is a promising candidate for binding to BSA. Its compact size and ease of synthesis make it an attractive option for incorporation into fusion constructs aimed at extending the half-life of therapeutic agents.

The peptide sequence THRPPMWSPVWP is known as a transferrin receptor-binding peptide (TfR-BP) and has been studied for its ability to bind to the transferrin receptor (TfR). This interaction can facilitate targeted delivery of therapeutic molecules or vaccines across cells expressing transferrin receptors, such as the blood-brain barrier or cancer cells.

HWRGWV: This hexameric peptide has been identified to bind the Fc region of IgG. It has been extensively characterized for its binding activity and has potential applications in antibody purification and targeted drug delivery.

The peptide sequence TRPPMWSPVWP is a transferrin receptor-binding peptide (TfR-BP) and has been studied for its ability to bind to the transferrin receptor (TfR). This interaction can facilitate targeted delivery of therapeutic molecules or vaccines across cells expressing transferrin receptors, such as the blood-brain barrier or cancer cells.

Short peptides that bind to transferrin or the Fc region of IgG offer promising strategies for enhancing the delivery and half-life of therapeutic agents. Adapting and validating these peptides for use in cattle could improve vaccine efficacy and therapeutic interventions in veterinary medicine.

Combining short peptides that bind albumin, transferrin, and Fc regions with an FMD (foot-and-mouth disease) vaccine antigen improves the vaccine's pharmacokinetics, delivery, and immune response.

Albumin binding prolongs the half-life of the antigen by utilizing albumin's natural long circulation time in the bloodstream (19-21 days in humans) by leveraging the albumin recycling pathway.

Albumin-binding peptides (ABPs) tether the antigen to albumin, leveraging the albumin recycling pathway mediated by the neonatal Fc receptor (FcRn). This protects the antigen from renal clearance and proteolytic degradation. Albumin conjugation can focus on prolonging half-life

Transferrin binding enables targeted delivery to cells expressing transferrin receptors (e.g., immune cells like macrophages and dendritic cells) and may enhance antigen uptake. Transferrin receptor-mediated endocytosis facilitates intracellular antigen delivery, potentially improving antigen processing and presentation for immune activation. Transferrin conjugation can specialize in targeted delivery to immune cells with transferrin receptors.

Fc binding enhances immune activation by engaging Fc receptors (FcRs) on immune cells and prolongs antigen half-life via FcRn recycling, similar to albumin. Fc-binding peptides mimic antibodies, enabling the antigen to activate antigen-presenting cells (APCs) and enhance adaptive immune responses. Fc conjugation can emphasize immune activation by engaging Fc receptors and facilitating better antigen presentation.

Including binding peptides improves the duration and localization of the antigen in the body, ensuring better exposure to the immune system. Transferrin- and Fc-mediated pathways could specifically target APCs, providing more efficient processing and presentation of the FMD antigen. Fc-binding elements also serve as an adjuvant, boosting immune activation.

Combining All Three Peptides in a Single Design Simultaneously addresses multiple challenges: half-life extension (albumin), targeting (transferrin), and immune activation (Fc) in a single molecule. This could reduce the need for boosters and improve overall vaccine efficacy. Transferrin and Fc binding pathways can synergize to enhance uptake by antigen-presenting cells, leading to stronger and more durable immune responses. A single vaccine formulation with multiple functions eliminates the need for separate doses targeting different mechanisms.

If the FMD antigen is inherently highly immunogenic, albumin or transferrin conjugation might be sufficient to extend its half-life and ensure uptake.

### DETAILED DESCRIPTION OF THE INVENTION

The method described in this patent application illustrates the formulation process to achieve a high-quality vaccine for FMD virus.

In one embodiment, the present invention relates to a method to formulate a universal vaccine against one or more serotypes and/or strains of an FMDV.

In one embodiment, the present invention provides immunogenic components to formulate different vaccines to ensure cross-protection against all or different serotypes or strains of a virus, such as FMDV.

In one embodiment, the present invention provides immunogenic components to formulate different vaccines to ensure total or cross-protection against different FMDV serotypes and/or strains in combination with one or more immunogenic components ensures a high protection that comprises cellular and humoral components of the immunological response.

In one embodiment, the universal vaccine of the present invention can specifically induce one or more targeted immune responses against all or different serotypes and/or strains of an FMDV.

### Polynucleotides Encoding Viral Peptides, Polypeptides, or Proteins in Different Types of Plasmids

One of the ordinary skills in art is readily recognizing that the present invention can be designed using any combination of polynucleotides derived from FMDV.

The present invention can also be designed using a combination of different FMDV polynucleotides. In one embodiment, these universal vaccines can comprise one or more polynucleotides that encode entire, partial, or variant sequences of FMDV proteins such as capsid proteins VP1, VP2, VP3 and VP4; or non-structural proteins such as 2A, 2B, 2C, 2D, 3A, 3B, 3C and 3D; or any polynucleotide sequences that encode the peptides of SEQ ID NO. 1-55 (Table 3) or the variants, fragments, homologous sequences, or functional analogs of such peptides.

These polynucleotide sequences can be cloned in any expression vector known in the art that can express these sequences in a eukaryotic cell environment. Suitable expression vectors can also be constructed using techniques of recombinant technology generally known in art. Examples of expression vectors with sequences encoding FMDV epitopes include, but are not limited to, pcDNA3.1/P1-2A3C3D, plasmid that comprise sequences encoding the viral structural protein precursor P1-2A (VP0, VP1, or VP3) and the non-structural proteins 3C and 3D; and the plasmids pCEIM and pCEIS that confer protection against FMDV in mice and swine due to VP1 DNA sequences cloned within them.

One of the ordinary skills in art is readily recognizing that the present invention can be designed using a combination of different recombinant peptides, polypeptides, and/or proteins derived from FMDV.

In one embodiment, the composition of the present invention comprises a combination of different FMDV-derived amino acid sequences. For example, one or more FMDV-derived amino acid sequences would encode the entire, partial, or variant sequences of FMDV capsid proteins such as VP1, VP2, VP3, and VP4; or non-structural proteins such as 2A, 2B, 2C, 2D, 3A, 3B, 3C and 3D.

In another embodiment, the genes of serotypes of FMD comprise AY593763, EF208770, EF208771, EF208772, EF208769, FJ755007, FJ755133, FJ755155, FJ755010, KY091291, KY091294, KY091290, KY091293, KY091296, KY091295, KY091297, AY593755, EF208777, EF208778, HQ116312, AF390646, KF561699, EF208756, AY593761, GU566064, KF561704, KF561698, KF561705, M10975, AY593780, AY593768, AJ306219, AY593766, EU553852, AY593795, DQ121109, DQ121119, DQ101240, FJ785230, DQ121116, AF390678, KY091304, JF739177, AY304994, AY390432, KY091300, FJ798151, KY091302, KY091301, M90381, AJ133357, M90376, KY091303, EU553893, AY593810, AJ294911, AJ294919, AJ294926, AF308157, AJ303500, AJ303502, AJ303503, AJ303509, AJ303501, AY593815, M55287, AJ004645, DQ834727, AJ303511, KY091280, AJ296327, DQ165074, DQ165077, DQ165073, KF561679, DQ165075, FJ798109, FJ798127, FJ798137, HM211075, FJ798141, AJ303488, AJ303485, AY593823, AF204276, AF292107, AJ539141, EU667451, KY091281, KY091284, KY091283, KY091282, KY091285, DQ164904, DQ164941, KM921876, KM921814, KY091288, AJ294910, DQ164925, KF561706, KF219690, AY593839, AY593846, AY593838, AY593845, KF219682, AF431711, AY593844, DQ009725, AY442010, AY442012, FJ798154, KX822796, KF219691, AY593848, KF219689, AF136607, EF134951, AY593847, AF367124, AY344505, AY343935, AF367139, AF479415, AF479410, AF367135, JX570616, DQ009737, AF367134, AJ251473, AY343941, AF367100, AY343969, AF479417, AY343963, AY343939, FJ798161, and AY343938, or a combination thereof.

Examples of suitable polypeptides derived from FMDV include but are not limited to, one or more native or recombinant peptides, polypeptides or proteins constructed entirely, partially, or mutated from the G-H loop of FMDV VP1, and a promiscuous artificial Th site derived from measles virus (UBITh1) which could give protection against FMD O1 Taiwan in pigs; native, or recombinant peptides, polypeptides and proteins derived entirely, partially or mutated from immunogenic epitopes in the VP1 (129-169), 3A (21-35), and 3D (346-370) proteins of the A/HuBWH/CHA/2009 strain of FMDV that elicits production of virus-neutralizing antibodies against serotype-A in cattle and guinea pigs. In one embodiment, the polypeptides are native or recombinant peptides, and polypeptides derived entirely, partially, or mutated from the hypervariable region of the GH loop, which can vary in length depending on the strain but is usually comprised between amino acids 135 and 160 of the VP1 capsid protein of FMDV. The hypervariable region of the GH loop of the VP1 protein contains significant epitopes. It is one of the major sites of phylogenetic diversity between FMDV strains since it represents an evasion mechanism from the pressure of the immune system for the diverging strains of FMDV. Indeed, antibodies developed by the host against this hypervariable loop specific to one strain neutralize antibodies against this strain but will not neutralize against another FMDV divergent strain. Therefore, the percentage of homology for different sequences is highly variable. Thus, a person skilled in the art can readily understand that the peptides and polypeptides derived from the GH loop and recognized as helpful for this invention are functional analogs and can have an amino acids sequence homology as low as 10% with the GH loop peptides of SEQ ID NO. 9-18 (Table 3).

In another embodiment, the amino acid sequences can be any of the peptides of SEQ ID NO. 1-55 (Table 3) or peptide variants, fragments, homologous sequences, or functional analogs derived from multiple sera of FMDV.

TABLE 3
Epitopes of FMDV

| # | Peptide | Sequence | Seqr | Source | Sera |
|---|---|---|---|---|---|
| 1 | Peptide A | TTTTGESADPVT | 1-12 | VP1 | Asia1 |
| 2 | Peptide B | TGESADPVTT | 4-13 | VP1 | AF/72 |
| 3 | Peptide_C | NYGGETQTARRLH | 17- | VP1 | Asia1 |
| | | | 29 | | |
| 4 | Peptide_D | ETQIQRRQHTDVSFIMDRFV | 21- | VP1 | O1Campos |
| | | | 40 | | |
| 5 | Peptide_E | QRRQHTDVSFIMDRFVK | 25- | VP1 | O1Kaufbeuren |
| | | | 41 | | |
| 6 | Peptide_F | RRQHTDVSF | 26- | VP1 | O/ISA/1/74 |
| | | | 34 | | |
| 7 | Peptide_G | LRTATYYFADLEVAV | 66- | VP1 | O/UKG/35/ |
| | | | 80 | | 2001 |
| 8 | Peptide_H | AYHKGPFTRL | 106- | VP1 | AF/72 |
| | | | 115 | | |
| 9 | Peptide_I | YSRNAVPNARGDLQVLAQKV | 136- | VP1 | O1Campos |
| | | A | 156 | | |
| 10 | Peptide_I_ | YTVSGLSRRGDLGSLAARVA | 136- | VP1 | A2001 |
| | A2001 | K | 156 | | |
| 11 | Peptide_I | YTGGSLPNVRGDLQVLAPKA | 136- | VP1 | O/SKR/JC/ |
| | Jincheon | A | 156 | | 2014 |
| 12 | Peptide_I_ | YGESDVTNVRGDLQVLAQK | 136- | VP1 | BHU/1/2013 |
| | Bhutan | AA | 156 | | |
| 13 | Peptide_I_ | YGENNVTNVRGDLQVLAQK | 136- | VP1 | SAU/3/2013 |
| | SAU | AA | 156 | | |
| 14 | Peptide_I_ | SKYSAPQNRRGDLGPLAARL | 136- | VP1 | A/HY/CHA/ |
| | China | A | 156 | | 2013 |
| 15 | Peptide_I_ | SKYSTPQTRRGDLGPLAARL | 136- | VP1 | A/VN/T11D/ |
| | Vietnam | A | 156 | | 2013 |
| 16 | Peptide_J | AVPNARGDLQVLAQKVARTL | 140- | VP1 | O1Campos |
| | | P | 160 | | |
| 17 | Peptide_J_ | SLPNVRGDLQVLAPKAARPL | 140- | VP1 | O/SKR/JC/ |
| | JinCheon | P | 160 | | 2014 |
| 18 | Peptide_K | LRGDLQVLAQKVARTL | 144- | VP1 | O1Kaufbeuren |
| | | | 159 | | |
| 19 | Peptide_L | RTLPTSFNY | 157- | VP1 | O1Campos |
| | | | 165 | | |
| 20 | Peptide_M | TTQDRRKQEIIAPEKQTL | 194- | VP1 | Asia1 |
| | | | 211 | | |
| 21 | Peptide_N | HKQKIVAPVKQTL | 201- | VP1 | O1Campos |
| | | | 213 | | |
| 22 | Peptide_O | EDAVSGPNTSG | 40- | VP2 | Asia1 |
| | | | 50 | | |
| 23 | Peptide_P | PFGHLTKLELPTDHH | 74- | VP2 | A10Holland |
| | | | 88 | | |
| 24 | Peptide_Q | YGKVSNPPRTSFPG | 26- | VP3 | Asia1 |
| | | | 39 | | |
| 25 | Peptide_R | DVSLAAKHMSNTYLS | 78- | VP3 | A10Holland |
| | | | 92 | | |
| 26 | Peptide_S | SIINNYYMQQYQNSMD | 20- | VP4 | A10Holland |
| | | | 35 | | |
| 27 | Peptide_T | YQNSMDTQLGDN | 30- | VP4 | Asia1 |
| | | | 41 | | |
| 28 | Peptide U | PFFFSDVRSNFSKLV | 1-15 | 2B | TAW/2/99 |
| 29 | Peptide_V | FFRSTPEDLERAEK | 140- | 2B | TAW/2/99 |
| | | | 153 | | |
| 30 | Peptide W | LKARDINDIFAILKN | 1-15 | 2C | TAW/2/99 |
| 31 | Peptide_X | SEEKFVTMTDLVPG | 36- | 2C | TAW/2/99 |
| | | | 50 | | |
| 32 | Peptide_Y | VTMTDLVPGILEKQR | 41- | 2C | TAW/2/99 |
| | | | 55 | | |
| 33 | Peptide_Z | YFLIEKGQHEAAIEF | 11- | 3A | O1Kaufbeuren |
| | | | 25 | | |
| 34 | Peptide_A1 | AAIEFFEGMVHDSIK | 21- | 3A | O1Campos |
| | | | 35 | | |
| 35 | Peptide_B1 | ERTLPGQKACDDVN | 126- | 3A | O1Kaufbeuren |
| | | | 139 | | |
| 36 | Peptide C1 | GPYAGPLETQKPLK | 1-14 | 3B | O1Kaufbeuren |
| 37 | Peptide D1 | PLERQKPLKVRAKL | 6-19 | 3B | O1Kaufbeuren |
| 38 | Peptide_E1 | GPYAGPMERQKPLK | 24- | 3B | O1Kaufbeuren |
| | | | 37 | | |
| 39 | Peptide_F1 | PMERQKPLKVKAKA | 29- | 3B | O1Kaufbeuren |
| | | | 42 | | |
| 40 | Peptide_G1 | QKPLKVKAKAPVVK | 33- | 3B | O1Kaufbeuren |
| | | | 46 | | |
| 41 | Peptide H1 | PVKKPVALKVKAKN | 52- | 3B | O1Kaufbeuren |
| | | | 65 | | |
| 42 | Peptide_I1 | NADVGRLIFSGEALT | 121- | 3C | O1Kaufbeuren |
| | | | 135 | | |
| 43 | Peptide_J1 | AVLAKDGADTFIVGT | 166- | 3C | O1Kaufbeuren |
| | | | 180 | | |
| 44 | Peptide_K1 | MRKTKLAPTVAHGVF | 16- | 3D | C-S8 |
| | | | 30 | | |
| 45 | Peptide_L1 | VLDEVIFSKHKGDTK | 51- | 3D | C-S8 |
| | | | 65 | | |
| 46 | Peptide_M1 | TANAPLSIYEAIKGVDGLDAM | 91- | 3D | C-S8 |
| | | EP | 115 | | |
| 47 | Peptide N1 | VDVLPVEHILYTRMMIGRFC | 181- | 3D | C-S8 |
| | | | 200 | | |
| 48 | Peptide_O1 | SATSIINTILNNIYV | 301- | 3D | C-S8 |
| | | | 315 | | |
| 49 | Peptide_Pl | VELDTYTMISYGDDI | 326- | 3D | C-S8 |
| | | | 340 | | |
| 50 | Peptide_Q1 | VVASDYDLDFEALKPHFKSL | 341- | 3D | C-S8 |
| | | | 360 | | |
| 51 | Peptide_R1 | YDLDFEALKPHFKSL | 346- | 3D | C-S8 |
| | | | 360 | | |
| 52 | Peptide_S1 | EALKPHFKSLGQTYT | 351- | 3D | C-S8 |
| | | | 365 | | |
| 53 | Peptide_T1 | HFKSLGQTYTPADKS | 356- | 3D | C-S8 |
| | | | 370 | | |
| 54 | Peptide_U1 | TDVTFLKRHFHMDYGTGFYK | 381- | 3D | C-S8 |
| | | | 400 | | |
| 55 | Peptide_V1 | KTLEAILSFARRGTI | 406- | 3D | C-S8 |
| | | | 420 | | |

This list contains various serotypes and strains of the commonly found Foot-and-Mouth Disease Virus (FMDV). The serotypes include Asia1, O, A, and C, with specific strains and regional identifiers. For the Asia1 serotype, multiple mentions highlight its relevance in studies. The O serotype is represented by strains such as O1Campos, O1Kaufbeuren, O/ISA/1/74, O/UK(G/35/2001, O/SKR/JC/2014, and O/SKR/JC/2014, indicating their geographic and temporal significance. The A serotype includes strains like A2001, A/HY/CHA/2013, A/VN/T11D/2013, and A10Holland, reflecting its diverse representation in global research. Strains such as BHU/1/2013 from Bhutan, SAU/3/2013 from Saudi Arabia, and TAW/2/99 from Taiwan are included to indicate their specific geographic origin. The C serotype is represented solely by the strain C-S8, which appears multiple times, emphasizing its utility in certain studies. Additionally, the repeated listing of strains such as O1Campos, O1Kaufbeuren, and TAW/2/99 suggests their extensive exposure. This comprehensive list reflects the diversity of FMDV serotypes and strains crucial for addressing the disease's epidemiology, prevention, and control.

These epitopes can be conjugated using a flexible linker such as GGGGSx3 (Sequence 56), resulting in a full protein sequence, as presented in Sequence 57. The sequence of three selected serum proteins to fuse with the antigen sequence include bovine IgG2 Fc (Sequence 58), bovine albumin (Sequence 59) and bovine transferrin (Sequence 60); Salmonella typhimurium flagellin (UniProt TD: P06179) or Escherichia coli flagellin (UniProt TD: P04949) are appropriate choice as adjuvants in recombinant expression (Sequence 61) connected to the antigen at one end. In contrast, the fusing proteins are connected at the other end of the antigen (Sequence 62-64) as a sequence of final antigens to produce immunity against FMD and treat ED. (Table 4).

In one embodiment, short peptides capable of binding albumin, Fc, and transferrin can be substituted for albumin, Fc, or transferrin as shown in Sequences 65-69, and these peptides conjugated by a linker Sequence 56, and the conjugated sequence then fused with the D complete protein antigen (Sequence 70).

TABLE 4
Antigen design peptides and complete antigen peptide sequences

| No | Peptide | Sequence | Length |
|---|---|---|---|
| 56 | Linker | GGGGSGGGGSGGGGS | 15 |
| 57 | Protein | TTTTGESADPVTGGGGSGGGGSGGGGSTGESADPVTTGGGGS | 1679 |
| | Full of | GGGGSGGGGSNYGGETQTARRLHGGGGSGGGGSGGGGSETQI | |
| | peptides | QRRQHTDVSFIMDRFVGGGGSGGGGSGGGGSQRRQHTDVSFI | |
| | linked | MDRFVKGGGGSGGGGSGGGGSRRQHTDVSFGGGGSGGGGSG | |
| | with | GGGSLRTATYYFADLEVAVGGGGSGGGGSGGGGSAYHKGPFT | |
| | G4Sx3 | RLGGGGSGGGGSGGGGSYSRNAVPNARGDLQVLAQKVAGGG | |
| | Linker | GSGGGGSGGGGSYTVSGLSRRGDLGSLAARVAKGGGGSGGGG | |
| | | SGGGGSYTGGSLPNVRGDLQVLAPKAAGGGGSGGGGSGGGG | |
| | | SYGESDVTNVRGDLQVLAQKAAGGGGSGGGGSGGGGSYGEN | |
| | | NVTNVRGDLQVLAQKAAGGGGSGGGGSGGGGSSKYSAPQNR | |
| | | RGDLGPLAARLAGGGGSGGGGSGGGGSSKYSTPQTRRGDLGP | |
| | | LAARLAGGGGSGGGGSGGGGSAVPNARGDLQVLAQKVARTLP | |
| | | GGGGSGGGGSGGGGSSLPNVRGDLQVLAPKAARPLPGGGGSG | |
| | | GGGSGGGGSLRGDLQVLAQKVARTLGGGGSGGGGSGGGGSR | |
| | | TLPTSFNYGGGGSGGGGSGGGGSTTQDRRKQEIIAPEKQTLGG | |
| | | GGSGGGGSGGGGSHKQKIVAPVKQTLGGGGSGGGGSGGGGSE | |
| | | DAVSGPNTSGGGGGSGGGGSGGGGSPFGHLTKLELPTDHHGG | |
| | | GGSGGGGSGGGGSYGKVSNPPRTSFPGGGGGSGGGGSGGGGS | |
| | | DVSLAAKHMSNTYLSGGGGSGGGGSGGGGSSIINNYYMQQY | |
| | | QNSMDGGGGSGGGGSGGGGSYQNSMDTQLGDNGGGGSGGG | |
| | | GSGGGGSPFFFSDVRSNFSKLVGGGGSGGGGSGGGGSFFRSTP | |
| | | EDLERAEKGGGGSGGGGSGGGGSLKARDINDIFAILKNGGGGS | |
| | | GGGGSGGGGSSEEKFVTMTDLVPGGGGGSGGGGSGGGGSVT | |
| | | MTDLVPGILEKQRGGGGSGGGGSGGGGSYFLIEKGQHEAAIEF | |
| | | GGGGSGGGGSGGGGSAAIEFFEGMVHDSIKGGGGSGGGGSGG | |
| | | GGSERTLPGQKACDDVNGGGGSGGGGSGGGGSGPYAGPLETQ | |
| | | KPLKGGGGSGGGGSGGGGSPLERQKPLKVRAKLGGGGSGGG | |
| | | GSGGGGSGPYAGPMERQKPLKGGGGSGGGGSGGGGSPMERQ | |
| | | KPLKVKAKAGGGGSGGGGSGGGGSQKPLKVKAKAPVVKGG | |
| | | GGSGGGGSGGGGSPVKKPVALKVKAKNGGGGSGGGGSGGGG | |
| | | SNADVGRLIFSGEALTGGGGSGGGGSGGGGSAVLAKDGADTFI | |
| | | VGTGGGGSGGGGSGGGGSMRKTKLAPTVAHGVFGGGGSGGG | |
| | | GSGGGGSVLDEVIFSKHKGDTKGGGGSGGGGSGGGGSTANAP | |
| | | LSIYEAIKGVDGLDAMEPGGGGSGGGGSGGGGSVDVLPVEHIL | |
| | | YTRMMIGRFCGGGGSGGGGSGGGGSSATSIINTILNNIYVGGG | |
| | | GSGGGGSGGGGSVELDTYTMISYGDDIGGGGSGGGGSGGGGS | |
| | | VVASDYDLDFEALKPHFKSLGGGGSGGGGSGGGGSYDLDFEA | |
| | | LKPHFKSLGGGGSGGGGSGGGGSEALKPHFKSLGQTYTGGGG | |
| | | SGGGGSGGGGSHFKSLGQTYTPADKSGGGGSGGGGSGGGGST | |
| | | DVTFLKRHFHMDYGTGFYKGGGGSGGGGSGGGGSKTLEAILS | |
| | | FARRGTIG | |
| 58 | Immunoglo- | MEQLRESGPSLVKPSQTLSLTCTVSGFSLNDNRVVWVRQPPGK | 448 |
| | bulin | APEWLGRIYSGGATHINPALKSRLSISKDNSKNEVSLSISDVRTE | |
| | gamma | DTATYMCGKEQTFNDGESSYIDVWGQGFRVIVSSASTTAPKVY | |
| | heavy | PLTSCCGDKSSSRVTLGCLVSSYMPEPVTVTWNSGALKSGVHT | |
| | chain | FPAVLQSSGLYSLSSMVTVPGSSSGQTFTCNVAHPASSTKVDKA | |
| | IgG2 | VGVSSDCSKPNNQHCVREPSVFIFPPKPKDTLMITGTPEVTCVV | |
| | [Bos | VNVGHDNPEVQFSWFVDDVEVHTARTKPREEQFNSTYRVVSA | |
| | taurus] | LPIQHQDWTGGKEFKCKVNIKGLSASIVRIISRSKGPAREPQVY | |
| | GenBa | VLDPPKEELSKSTVSLTCMVIGFYPEDVDVEWQRDRQTESEDK | |
| | nk: | YRTTPPQLDADRSYFLYSKLRVDRNSWQRGDTYTCVVMHEAL | |
| | AQT27056.1 | HNHYMQKSTSKSAGK | |
| 59 | Albumin | MKWVTFISLLLLFSSAYSRGVFRRDTHKSEIAHRFKDLGEEHFK | 607 |
| | [Bos | GLVLIAFSQYLQQCPFDEHVKLVNELTEFAKTCVADESHAGCE | |
| | taurus] | KSLHTLFGDELCKVASLRETYGDMADCCEKQEPERNECFLSHK | |
| | GenBa | DDSPDLPKLKPDPNTLCDEFKADEKKFWGKYLYEIARRHPYFY | 704 |
| | nk: | APELLYYANKYNGVFQECCQAEDKGACLLPKIETMREKVLASS | |
| | AAA51411.1 | ARQRLRCASIQKFGERALKAWSVARLSQKFPKAEFVEVTKLVT | |
| | | DLTKVHKECCHGDLLECADDRADLAKYICDNQDTISSKLKEC | |
| | | CDKPLLEKSHCIAEVEKDAIPENLPPLTADFAEDKDVCKNYQE | |
| | | AKDAFLGSFLYEYSRRHPEYAVSVLLRLAKEYEATLEECCAKD | |
| | | DPHACYSTVFDKLKHLVDEPQNLIKQNCDQFEKLGEYGFQNA | |
| | | LIVRYTRKVPQVSTPTLVEVSRSLGKVGTRCCTKPESERMPCTE | |
| | | DYLSLILNRLCVLHEKTPVSEKVTKCCTESLVNRRPCFSALTPD | |
| | | ETYVPKAFDEKLFTFHADICTLPDTEKQIKKQTALVELLKHKPK | |
| | | ATEEQLKTVMENFVAFVDKCCAADDKEACFAVEGPKLVVSTQ | |
| | | TALA | |
| 60 | transferrin | MRPAVRALLACAVLGLCLADPERTVRWCTISTHEANKCASFRE | |
| | [Bos | NVLRILESGPFVSCVKKTSHMDCIKAISNNEADAVTLDGGLVY | |
| | taurus] | EAGLKPNNLKPVVAEFHGTKDNPQTHYYAVAVVKKDTDFKLN | |
| | GenBa | ELRGKKSCHTGLGRSAGWNIPMAKLYKELPDPQESIQRAAANF | |
| | nk: | FSASCVPCADQSSFPKLCQLCAGKGTDKCACSNHEPYFGYSGA | |
| | AAA96735.1 | FKCLMEGAGDVAFVKHSTVFDNLPNPEDRKNYELLCGDNTRK | |
| | | SVDDYQECYLAMVPSHAVVARTVGGKEDVIWELLNHAQEHFG | |
| | | KDKPDNFQLFQSPHGKDLLFKDSADGFLKIPSKMDFELYLGYE | |
| | | YVTALQNLRESKPPDSSKDECMVKWCAIGHQERTKCDRWSGF | |
| | | SGGAIECETAENTEECIAKIMKGEADAMSLDGGYLYIAGKCGL | |
| | | VPVLAENYKTEGESCKNTPEKGYLAVAVVKTSDANINWNNLK | |
| | | DKKSCHTAVDRTAGWNIPMGLLYSKINNCKFDEFFSAGCAPGS | |
| | | PRNSSLCALCIGSEKGTGKECVPNSNERYYGYTGAFRCLVEKG | |
| | | DVAFVKDQTVIQNTDGNNNEAWAKNLKKENFEVLCKDGTRKP | |
| | | VTDAENCHLARGPNHAVVSRKDKATCVEKILNKQQDDFGKSV | |
| | | TDCTSNFCLFQSNSKDLLFRDDTKCLASIAKKTYDSYLGDDYV | |
| | | RAMTNLRQCSTSKLLEACTFHKP | |
| 61 | P06179 | MAQVINTNSLSLLTQNNLNKSQSALGTAIERLSSGLRINSAKDD | 495 |
| | FLIC | AAGQAIANRFTANIKGLTQASRNANDGISIAQTTEGALNEINNN | |
| | SALTY | LQRVRELAVQSANSTNSQSDLDSIQAEITQRLNEIDRVSGQTQF | |
| | | NGVKVLAQDNTLTIQVGANDGETIDIDLKQINSQTLGLDTLNV | |
| | | QQKYKVSDTAATVTGYADTTIALDNSTFKASATGLGGTDQKID | |
| | | GDLKFDDTTGKYYAKVTVTGGTGKDGYYEVSVDKTNGEVTL | |
| | | AGGATSPLTGGLPATATEDVKNVQVANADLTEAKAALTAAGVT | |
| | | GTASVVKMSYTDNNGKTIDGGLAVKVGDDYYSATQNKDGSISI | |
| | | NTTKYTADDGTSKTALNKLGGADGKTEVVSIGGKTYAASKAE | |
| | | GHNFKAQPDLAEAAATTTENPLQKIDAALAQVDTLRSDLGAV | |
| | | QNRFNSAITNLGNTVNNLTSARSRIEDSDYATEVSNMSRAQILQ | |
| | | QAGTSVLAQANQVPQNVLSLLR | |
| 62 | P06179· | MAQVINTNSLSLLTQNNLNKSQSALGTAIERLSSGLRINSAKDD | 2651 |
| | FLIC | AAGQAIANRFTANIKGLTQASRNANDGISIAQTTEGALNEINNN | |
| | SALTY+ | LQRVRELAVQSANSTNSQSDLDSIQAEITQRLNEIDRVSGQTQF | |
| | linker+ | NGVKVLAQDNTLTIQVGANDGETIDIDLKQINSQTLGLDTLNV | |
| | Full | QQKYKVSDTAATVTGYADTTIALDNSTFKASATGLGGTDQKID | |
| | Protein+ | GDLKFDDTTGKYYAKVTVTGGTGKDGYYEVSVDKTNGEVTL | |
| | linker+ | AGGATSPLTGGLPATATEDVKNVQVANADLTEAKAALTAAGVT | |
| | IgG2 | GTASVVKMSYTDNNGKTIDGGLAVKVGDDYYSATQNKDGSISI | |
| | Fc | NTTKYTADDGTSKTALNKLGGADGKTEVVSIGGKTYAASKAE | |
| | | GHNFKAQPDLAEAAATTTENPLQKIDAALAQVDTLRSDLGAV | |
| | | QNRFNSAITNLGNTVNNLTSARSRIEDSDYATEVSNMSRAQILQ | |
| | | QAGTSVLAQANQVPQNVLSLLRGGGGSGGGGSGGGGSTTTTG | |
| | | ESADPVTGGGGSGGGGSGGGGSTGESADPVTTGGGGSGGGGS | |
| | | GGGGSNYGGETQTARRLHGGGGSGGGGSGGGGSETQIQRRQH | |
| | | TDVSFIMDRFVGGGGSGGGGSGGGGSQRRQHTDVSFIMDRFV | |
| | | KGGGGSGGGGSGGGGSRRQHTDVSFGGGGSGGGGSGGGGSL | |
| | | RTATYYFADLEVAVGGGGSGGGGSGGGGSAYHKGPFTRLGGG | |
| | | GSGGGGSGGGGSYSRNAVPNARGDLQVLAQKVAGGGGSGGG | |
| | | GSGGGGSYTVSGLSRRGDLGSLAARVAKGGGGSGGGGSGGGG | |
| | | SYTGGSLPNVRGDLQVLAPKAAGGGGSGGGGSGGGGSYGES | |
| | | DVTNVRGDLQVLAQKAAGGGGSGGGGSGGGGSYGENNVTN | |
| | | VRGDLQVLAQKAAGGGGSGGGGSGGGGSSKYSAPQNRRGDL | |
| | | GPLAARLAGGGGSGGGGSGGGGSSKYSTPQTRRGDLGPLAAR | |
| | | LAGGGGSGGGGSGGGGSAVPNARGDLQVLAQKVARTLPGGG | |
| | | GSGGGGSGGGGSSLPNVRGDLQVLAPKAARPLPGGGGSGGGG | |
| | | SGGGGSLRGDLQVLAQKVARTLGGGGSGGGGSGGGGSRTLPT | |
| | | SFNYGGGGSGGGGSGGGGSTTQDRRKQEIIAPEKQTLGGGGSG | |
| | | GGGSGGGGSHKQKIVAPVKQTLGGGGSGGGGSGGGGSEDAVS | |
| | | GPNTSGGGGGSGGGGSGGGGSPFGHLTKLELPTDHHGGGGSG | |
| | | GGGSGGGGSYGKVSNPPRTSFPGGGGGSGGGGSGGGGSDVSL | |
| | | AAKHMSNTYLSGGGGSGGGGSGGGGSSIINNYYMQQYQNSM | |
| | | DGGGGSGGGGSGGGGSYQNSMDTQLGDNGGGGSGGGGSGG | |
| | | GGSPFFFSDVRSNFSKLVGGGGSGGGGSGGGGSFFRSTPEDLER | |
| | | AEKGGGGSGGGGSGGGGSLKARDINDIFAILKNGGGGSGGGG | |
| | | SGGGGSSEEKFVTMTDLVPGGGGGSGGGGSGGGGSVTMTDLV | |
| | | PGILEKQRGGGGSGGGGSGGGGSYFLIEKGQHEAAIEFGGGGS | |
| | | GGGGSGGGGSAAIEFFEGMVHDSIKGGGGSGGGGSGGGGSER | |
| | | TLPGQKACDDVNGGGGSGGGGSGGGGSGPYAGPLETQKPLKG | |
| | | GGGSGGGGSGGGGSPLERQKPLKVRAKLGGGGSGGGGSGGG | |
| | | GSGPYAGPMERQKPLKGGGGSGGGGSGGGGSPMERQKPLKV | |
| | | KAKAGGGGSGGGGSGGGGSQKPLKVKAKAPVVKGGGGSGG | |
| | | GGSGGGGSPVKKPVALKVKAKNGGGGSGGGGSGGGGSNADV | |
| | | GRLIFSGEALTGGGGSGGGGSGGGGSAVLAKDGADTFIVGTGG | |
| | | GGSGGGGSGGGGSMRKTKLAPTVAHGVFGGGGSGGGGSGGG | |
| | | GSVLDEVIFSKHKGDTKGGGGSGGGGSGGGGSTANAPLSIYEA | |
| | | IKGVDGLDAMEPGGGGSGGGGSGGGGSVDVLPVEHILYTRM | |
| | | MIGRFCGGGGSGGGGSGGGGSSATSIINTILNNIYVGGGGSGGG | |
| | | GSGGGGSVELDTYTMISYGDDIGGGGSGGGGSGGGGSVVASD | |
| | | YDLDFEALKPHFKSLGGGGSGGGGSGGGGSYDLDFEALKPHF | |
| | | KSLGGGGSGGGGSGGGGSEALKPHFKSLGQTYTGGGGSGGGG | |
| | | SGGGGSHFKSLGQTYTPADKSGGGGSGGGGSGGGGSTDVTFL | |
| | | KRHFHMDYGTGFYKGGGGSGGGGSGGGGSKTLEAILSFARRG | |
| | | TIGGGGSGGGGSGGGGSMEQLRESGPSLVKPSQTLSLTCTVSGF | |
| | | SLNDNRVVWVRQPPGKAPEWLGRIYSGGATHINPALKSRLSIS | |
| | | KDNSKNEVSLSISDVRTEDTATYMCGKEQTFNDGESSYIDVWG | |
| | | QGFRVIVSSASTTAPKVYPLTSCCGDKSSSRVTLGCLVSSYMPE | |
| | | PVTVTWNSGALKSGVHTFPAVLQSSGLYSLSSMVTVPGSSSGQ | |
| | | TFTCNVAHPASSTKVDKAVGVSSDCSKPNNQHCVREPSVFIFPP | |
| | | KPKDTLMITGTPEVTCVVVNVGHDNPEVQFSWFVDDVEVHTA | |
| | | RTKPREEQFNSTYRVVSALPIQHQDWTGGKEFKCKVNIKGLSA | |
| | | SIVRIISRSKGPAREPQVYVLDPPKEELSKSTVSLTCMVIGFYPE | |
| | | DVDVEWQRDRQTESEDKYRTTPPQLDADRSYFLYSKLRVDRN | |
| | | SWQRGDTYTCVVMHEALHNHYMQKSTSKSAGK | |
| 63 | P06179· | MAQVINTNSLSLLTQNNLNKSQSALGTAIERLSSGLRINSAKDD | 2188 |
| | FLIC | AAGQAIANRFTANIKGLTQASRNANDGISIAQTTEGALNEINNN | |
| | SALTY+ | LQRVRELAVQSANSTNSQSDLDSIQAEITQRLNEIDRVSGQTQF | |
| | linker+ | NGVKVLAQDNTLTIQVGANDGETIDIDLKQINSQTLGLDTLNV | |
| | Full | QQKYKVSDTAATVTGYADTTIALDNSTFKASATGLGGTDQKID | |
| | Protein+ | GDLKFDDTTGKYYAKVTVTGGTGKDGYYEVSVDKINGEVTL | |
| | linker+ | AGGATSPLTGGLPATATEDVKNVQVANADLTEAKAALTAAGVT | |
| | Transf | GTASVVKMSYTDNNGKTIDGGLAVKVGDDYYSATQNKDGSISI | |
| | errin | NTTKYTADDGTSKTALNKLGGADGKTEVVSIGGKTYAASKAE | |
| | | GHNFKAQPDLAEAAATTTENPLQKIDAALAQVDTLRSDLGAV | |
| | | QNRFNSAITNLGNTVNNLTSARSRIEDSDYATEVSNMSRAQILQ | |
| | | QAGTSVLAQANQVPQNVLSLLRTTTTGESADPVTGGGGSGGG | |
| | | GSGGGGSTGESADPVTTGGGGSGGGGSGGGGSNYGGETQTAR | |
| | | RLHGGGGSGGGGSGGGGSETQIQRRQHTDVSFIMDRFVGGGG | |
| | | SGGGGSGGGGSQRRQHTDVSFIMDRFVKGGGGSGGGGSGGG | |
| | | GSRRQHTDVSFGGGGSGGGGSGGGGSLRTATYYFADLEVAVG | |
| | | GGGSGGGGSGGGGSAYHKGPFTRLGGGGSGGGGSGGGGSYS | |
| | | RNAVPNARGDLQVLAQKVAGGGGSGGGGSGGGGSYTVSGLS | |
| | | RRGDLGSLAARVAKGGGGSGGGGSGGGGSYTGGSLPNVRGDL | |
| | | QVLAPKAAGGGGSGGGGSGGGGSYGESDVTNVRGDLQVLAQ | |
| | | KAAGGGGSGGGGSGGGGSYGENNVTNVRGDLQVLAQKAAG | |
| | | GGGSGGGGSGGGGSSKYSAPQNRRGDLGPLAARLAGGGGSG | |
| | | GGGSGGGGSSKYSTPQTRRGDLGPLAARLAGGGGSGGGGSGG | |
| | | GGSAVPNARGDLQVLAQKVARTLPGGGGSGGGGSGGGGSSLP | |
| | | NVRGDLQVLAPKAARPLPGGGGSGGGGSGGGGSLRGDLQVL | |
| | | AQKVARTLGGGGSGGGGSGGGGSRTLPTSFNYGGGGSGGGGS | |
| | | GGGGSTTQDRRKQEIIAPEKQTLGGGGSGGGGSGGGGSHKQKI | |
| | | VAPVKQTLGGGGSGGGGSGGGGSEDAVSGPNTSGGGGGSGGG | |
| | | GSGGGGSPFGHLTKLELPTDHHGGGGSGGGGSGGGGSYGKVS | |
| | | NPPRTSFPGGGGGSGGGGSGGGGSDVSLAAKHMSNTYLSGGG | |
| | | GSGGGGSGGGGSSIINNYYMQQYQNSMDGGGGSGGGGSGGG | |
| | | GSYQNSMDTQLGDNGGGGSGGGGSGGGGSPFFFSDVRSNFSK | |
| | | LVGGGGSGGGGSGGGGSFFRSTPEDLERAEKGGGGSGGGGSG | |
| | | GGGSLKARDINDIFAILKNGGGGSGGGGSGGGGSSEEKFVTMT | |
| | | DLVPGGGGGSGGGGSGGGGSVTMTDLVPGILEKQRGGGGSGG | |
| | | GGSGGGGSYFLIEKGQHEAAIEFGGGGSGGGGSGGGGSAAIEF | |
| | | FEGMVHDSIKGGGGSGGGGSGGGGSERTLPGQKACDDVNGG | |
| | | GGSGGGGSGGGGSGPYAGPLETQKPLKGGGGSGGGGSGGGGS | |
| | | PLERQKPLKVRAKLGGGGSGGGGSGGGGSGPYAGPMERQKPL | |
| | | KGGGGSGGGGSGGGGSPMERQKPLKVKAKAGGGGSGGGGSG | |
| | | GGGSQKPLKVKAKAPVVKGGGGSGGGGSGGGGSPVKKPVAL | |
| | | KVKAKNGGGGSGGGGSGGGGSNADVGRLIFSGEALTGGGGS | |
| | | GGGGSGGGGSAVLAKDGADTFIVGTGGGGSGGGGSGGGGSM | |
| | | RKTKLAPTVAHGVFGGGGSGGGGSGGGGSVLDEVIFSKHKGD | |
| | | TKGGGGSGGGGSGGGGSTANAPLSIYEAIKGVDGLDAMEPGG | |
| | | GGSGGGGSGGGGSVDVLPVEHILYTRMMIGRFCGGGGSGGGG | |
| | | SGGGGSSATSIINTILNNIYVGGGGSGGGGSGGGGSVELDTYTM | |
| | | ISYGDDIGGGGSGGGGSGGGGSVVASDYDLDFEALKPHFKSLG | |
| | | GGGSGGGGSGGGGSYDLDFEALKPHFKSLGGGGSGGGGSGG | |
| | | GGSEALKPHFKSLGQTYTGGGGSGGGGSGGGGSHFKSLGQTY | |
| | | TPADKSGGGGSGGGGSGGGGSTDVTFLKRHFHMDYGTGFYK | |
| | | GGGGSGGGGSGGGGSKTLEAILSFARRGTIGGGGSGGGGSGG | |
| | | GGS | |
| 64 | P06179· | MAQVINTNSLSLLTQNNLNKSQSALGTAIERLSSGLRINSAKDD | 3499 |
| | FLIC | AAGQAIANRFTANIKGLTQASRNANDGISIAQTTEGALNEINNN | |
| | SALTY+ | LQRVRELAVQSANSTNSQSDLDSIQAEITQRLNEIDRVSGQTQF | |
| | linker+ | NGVKVLAQDNTLTIQVGANDGETIDIDLKQINSQTLGLDTLNV | |
| | Full | QQKYKVSDTAATVTGYADTTIALDNSTFKASATGLGGTDQKID | |
| | Protein+ | GDLKFDDTTGKYYAKVTVTGGTGKDGYYEVSVDKTNGEVTL | |
| | linker+ | AGGATSPLTGGLPATATEDVKNVQVANADLTEAKAALTAAGVT | |
| | Albumin | GTASVVKMSYTDNNGKTIDGGLAVKVGDDYYSATQNKDGSISI | |
| | | NTTKYTADDGTSKTALNKLGGADGKTEVVSIGGKTYAASKAE | |
| | | GHNFKAQPDLAEAAATTTENPLQKIDAALAQVDTLRSDLGAV | |
| | | QNRFNSAITNLGNTVNNLTSARSRIEDSDYATEVSNMSRAQILQ | |
| | | QAGTSVLAQANQVPQNVLSLLRTTTTGESADPVTGGGGSGGG | |
| | | GSGGGGSTGESADPVTTGGGGSGGGGSGGGGSNYGGETQTAR | |
| | | RLHGGGGSGGGGSGGGGSETQIQRRQHTDVSFIMDRFVGGGG | |
| | | SGGGGSGGGGSQRRQHTDVSFIMDRFVKGGGGSGGGGSGGG | |
| | | GSRRQHTDVSFGGGGSGGGGSGGGGSLRTATYYFADLEVAVG | |
| | | GGGSGGGGSGGGGSAYHKGPFTRLGGGGSGGGGSGGGGSYS | |
| | | RNAVPNARGDLQVLAQKVAGGGGSGGGGSGGGGSYTVSGLS | |
| | | RRGDLGSLAARVAKGGGGSGGGGSGGGGSYTGGSLPNVRGDL | |
| | | QVLAPKAAGGGGSGGGGSGGGGSYGESDVTNVRGDLQVLAQ | |
| | | KAAGGGGSGGGGSGGGGSYGENNVTNVRGDLQVLAQKAAG | |
| | | GGGSGGGGSGGGGSSKYSAPQNRRGDLGPLAARLAGGGGSG | |
| | | GGGSGGGGSSKYSTPQTRRGDLGPLAARLAGGGGSGGGGSGG | |
| | | GGSAVPNARGDLQVLAQKVARTLPGGGGSGGGGSGGGGSSLP | |
| | | NVRGDLQVLAPKAARPLPGGGGSGGGGSGGGGSLRGDLQVL | |
| | | AQKVARTLGGGGGGGGSGGGGSRTLPTSFNYGGGGSGGGGS | |
| | | GGGGSTTQDRRKQEIIAPEKQTLGGGGSGGGGSGGGGSHKQKI | |
| | | VAPVKQTLGGGGSGGGGSGGGGSEDAVSGPNTSGGGGGSGGG | |
| | | GSGGGGSPFGHLTKLELPTDHHGGGGSGGGGSGGGGSYGKVS | |
| | | NPPRTSFPGGGGGSGGGGSGGGGSDVSLAAKHMSNTYLSGGG | |
| | | GSGGGGSGGGGSSIINNYYMQQYQNSMDGGGGSGGGGSGGG | |
| | | GSYQNSMDTQLGDNGGGGSGGGGSGGGGSPFFFSDVRSNFSK | |
| | | LVGGGGSGGGGSGGGGSFFRSTPEDLERAEKGGGGSGGGGSG | |
| | | GGGSLKARDINDIFAILKNGGGGSGGGGSGGGGSSEEKFVTMT | |
| | | DLVPGGGGGSGGGGSGGGGSVTMTDLVPGILEKQRGGGGSGG | |
| | | GGSGGGGSYFLIEKGQHEAAIEFGGGGSGGGGSGGGGSAAIEF | |
| | | FEGMVHDSIKGGGGSGGGGSGGGGSERTLPGQKACDDVNGG | |
| | | GGSGGGGSGGGGSGPYAGPLETQKPLKGGGGSGGGGSGGGGS | |
| | | PLERQKPLKVRAKLGGGGSGGGGSGGGGSGPYAGPMERQKPL | |
| | | KGGGGSGGGGSGGGGSPMERQKPLKVKAKAGGGGSGGGGSG | |
| | | GGGSQKPLKVKAKAPVVKGGGGSGGGGSGGGGSPVKKPVAL | |
| | | KVKAKNGGGGSGGGGSGGGGSNADVGRLIFSGEALTGGGGS | |
| | | GGGGSGGGGSAVLAKDGADTFIVGTGGGGSGGGGSGGGGSM | |
| | | RKTKLAPTVAHGVFGGGGSGGGGSGGGGSVLDEVIFSKHKGD | |
| | | TKGGGGSGGGGSGGGGSTANAPLSIYEAIKGVDGLDAMEPGG | |
| | | GGSGGGGSGGGGSVDVLPVEHILYTRMMIGRFCGGGGSGGGG | |
| | | SGGGGSSATSIINTILNNIYVGGGGSGGGGSGGGGSVELDTYTM | |
| | | ISYGDDIGGGGSGGGGSGGGGSVVASDYDLDFEALKPHFKSLG | |
| | | GGGSGGGGSGGGGSYDLDFEALKPHFKSLGGGGSGGGGSGG | |
| | | GGSEALKPHFKSLGQTYTGGGGSGGGGSGGGGSHFKSLGQTY | |
| | | TPADKSGGGGSGGGGSGGGGSTDVTFLKRHFHMDYGTGFYK | |
| | | GGGGGGGGSGGGGSKTLEAILSFARRGTIGGGGSGGGGSGG | |
| | | GGSMKWVTFISLLLLFSSAYSRGVFRRDTHKSEIAHRFKDLGEE | |
| | | HFKGLVLIAFSQYLQQCPFDEHVKLVNELTEFAKTCVADESHA | |
| | | GCEKSLHTLFGDELCKVASLRETYGDMADCCEKQEPERNECFL | |
| | | SHKDDSPDLPKLKPDPNTLCDEFKADEKKFWGKYLYEIARRHP | |
| | | YFYAPELLYYANKYNGVFQECCQAEDKGACLLPKIETMREKV | |
| | | LASSARQRLRCASIQKFGERALKAWSVARLSQKFPKAEFVEVT | |
| | | KLVTDLTKVHKECCHGDLLECADDRADLAKYICDNQDTISSKL | |
| | | KECCDKPLLEKSHCIAEVEKDAIPENLPPLTADFAEDKDVCKNY | |
| | | QEAKDAFLGSFLYEYSRRHPEYAVSVLLRLAKEYEATLEECCA | |
| | | KDDPHACYSTVFDKLKHLVDEPQNLIKQNCDQFEKLGEYGFQ | |
| | | NALIVRYTRKVPQVSTPTLVEVSRSLGKVGTRCCTKPESERMP | |
| | | CTEDYLSLILNRLCVLHEKTPVSEKVTKCCTESLVNRRPCFSAL | |
| | | TPDETYVPKAFDEKLFTFHADICTLPDTEKQIKKQTALVELLKH | |
| | | KPKATEEQLKTVMENFVAFVDKCCAADDKEACFAVEGPKLVV | |
| | | STQTALAMRPAVRALLACAVLGLCLADPERTVRWCTISTHEAN | |
| | | KCASFRENVLRILESGPFVSCVKKTSHMDCIKAISNNEADAVTL | |
| | | DGGLVYEAGLKPNNLKPVVAEFHGTKDNPQTHYYAVAVVKKD | |
| | | TDFKLNELRGKKSCHTGLGRSAGWNIPMAKLYKELPDPQESIQ | |
| | | RAAANFFSASCVPCADQSSFPKLCQLCAGKGTDKCACSNHEP | |
| | | YFGYSGAFKCLMEGAGDVAFVKHSTVFDNLPNPEDRKNYELL | |
| | | CGDNTRKSVDDYQECYLAMVPSHAVVARTVGGKEDVIWELL | |
| | | NHAQEHFGKDKPDNFQLFQSPHGKDLLFKDSADGFLKIPSKM | |
| | | DFELYLGYEYVTALQNLRESKPPDSSKDECMVKWCAIGHQER | |
| | | TKCDRWSGFSGGAIECETAENTEECIAKIMKGEADAMSLDGGY | |
| | | LYIAGKCGLVPVLAENYKTEGESCKNTPEKGYLAVAVVKTSDA | |
| | | NINWNNLKDKKSCHTAVDRTAGWNIPMGLLYSKINNCKFDEFF | |
| | | SAGCAPGSPRNSSLCALCIGSEKGTGKECVPNSNERYYGYTGA | |
| | | FRCLVEKGDVAFVKDQTVIQNTDGNNNEAWAKNLKKENFEVL | |
| | | CKDGTRKPVTDAENCHLARGPNHAVVSRKDKATCVEKILNKQ | |
| | | QDDFGKSVTDCTSNFCLFQSNSKDLLFRDDTKCLASIAKKTYD | |
| | | SYLGDDYVRAMTNLRQCSTSKLLEACTFHKP | |
| 65 | Short_ | DDDDKNQTPGNARILQTMKGL | 21 |
| | synthetic_ | | |
| | albumin | | |
| | peptide 1 | | |
| 66 | Short_ | KQNQQSSSEQDYSKKKK | 17 |
| | synthetic_ | | |
| | albumin | | |
| | peptide 2 | | |
| 67 | Short_ | DICLPRWGCLW | 11 |
| | synthetic_ | | |
| | albumin | | |
| | peptide 3 | | |
| 68 | Short_ | DAHK | 4 |
| | synthetic_ | | |
| | albumin | | |
| | peptide 4 | | |
| 69 | Short_ | THRPPMWSPVWP | 12 |
| | synthetic_ | | |
| | peptide_ | | |
| | transferrin | | |
| 69 | Short_ | HWRGWV | 6 |
| | synthetic_ | | |
| | peptide_ | | |
| | Fc | | |
| 70 | Conjugate_ | DICLPRWGCLWGGGGSGGGGSGGGGSTHRPPMWSPVWPGGG | 1753 |
| | of_albumin_ | GSGGGGSGGGGSHWRGWVGGGGSGGGGSGGGGSTTTTGESA | |
| | Fc_and_ | DPVTGGGGSGGGGSGGGGSTGESADPVTTGGGGSGGGGSGG | |
| | transferrin_ | GGSNYGGETQTARRLHGGGGSGGGGSGGGGSETQIQRRQHTD | |
| | synthetic_ | VSFIMDRFVGGGGSGGGGSGGGGSQRRQHTDVSFIMDRFVKG | |
| | peptide_ | GGGSGGGGSGGGGSRRQHTDVSFGGGGSGGGGSGGGGSLRT | |
| | with_ | ATYYFADLEVAVGGGGSGGGGSGGGGSAYHKGPFTRLGGGGS | |
| | linkers_ | GGGGSGGGGSYSRNAVPNARGDLQVLAQKVAGGGGSGGGGS | |
| | fused_ | GGGGSYTVSGLSRRGDLGSLAARVAKGGGGSGGGGSGGGGSY | |
| | with_full_ | TGGSLPNVRGDLQVLAPKAAGGGGSGGGGSGGGGSYGESDV | |
| | antigen_ | TNVRGDLQVLAQKAAGGGGSGGGGSGGGGSYGENNVTNVR | |
| | sequence | GDLQVLAQKAAGGGGSGGGGSGGGGSSKYSAPQNRRGDLGP | |
| | | LAARLAGGGGSGGGGSGGGGSSKYSTPQTRRGDLGPLAARLA | |
| | | GGGGSGGGGSGGGGSAVPNARGDLQVLAQKVARTLPGGGGS | |
| | | GGGGSGGGGSSLPNVRGDLQVLAPKAARPLPGGGGSGGGGSG | |
| | | GGGSLRGDLQVLAQKVARTLGGGGSGGGGSGGGGSRTLPTSF | |
| | | NYGGGGSGGGGSGGGGSTTQDRRKQEIIAPEKQTLGGGGSGG | |
| | | GGSGGGGSHKQKIVAPVKQTLGGGGSGGGGSGGGGSEDAVSG | |
| | | PNTSGGGGGSGGGGSGGGGSPFGHLTKLELPTDHHGGGGSGG | |
| | | GGSGGGGSYGKVSNPPRTSFPGGGGGSGGGGSGGGGSDVSLA | |
| | | AKHMSNTYLSGGGGSGGGGSGGGGSSIINNYYMQQYQNSMD | |
| | | GGGGSGGGGSGGGGSYQNSMDTQLGDNGGGGSGGGGSGGG | |
| | | GSPFFFSDVRSNFSKLVGGGGSGGGGSGGGGSFFRSTPEDLERA | |
| | | EKGGGGSGGGGSGGGGSLKARDINDIFAILKNGGGGSGGGGS | |
| | | GGGGSSEEKFVTMTDLVPGGGGGSGGGGSGGGGSVTMTDLVP | |
| | | GILEKQRGGGGSGGGGSGGGGSYFLIEKGQHEAAIEFGGGGSG | |
| | | GGGSGGGGSAAIEFFEGMVHDSIKGGGGSGGGGSGGGGSERT | |
| | | LPGQKACDDVNGGGGSGGGGSGGGGSGPYAGPLETQKPLKG | |
| | | GGGSGGGGSGGGGSPLERQKPLKVRAKLGGGGSGGGGSGGG | |
| | | GSGPYAGPMERQKPLKGGGGSGGGGSGGGGSPMERQKPLKV | |
| | | KAKAGGGGSGGGGSGGGGSQKPLKVKAKAPVVKGGGGSGG | |
| | | GGSGGGGSPVKKPVALKVKAKNGGGGSGGGGSGGGGSNADV | |
| | | GRLIFSGEALTGGGGSGGGGSGGGGSAVLAKDGADTFIVGTGG | |
| | | GGSGGGGSGGGGSMRKTKLAPTVAHGVFGGGGSGGGGSGGG | |
| | | GSVLDEVIFSKHKGDTKGGGGSGGGGSGGGGSTANAPLSIYEA | |
| | | IKGVDGLDAMEPGGGGSGGGGSGGGGSVDVLPVEHILYTRM | |
| | | MIGRFCGGGGSGGGGSGGGGSSATSIINTILNNIYVGGGGSGGG | |
| | | GSGGGGSVELDTYTMISYGDDIGGGGSGGGGSGGGGSVVASD | |
| | | YDLDFEALKPHFKSLGGGGSGGGGSGGGGSYDLDFEALKPHF | |
| | | KSLGGGGSGGGGSGGGGSEALKPHFKSLGQTYTGGGGSGGGG | |
| | | SGGGGSHFKSLGQTYTPADKSGGGGSGGGGSGGGGSTDVTFL | |
| | | KRHFHMDYGTGFYKGGGGSGGGGSGGGGSKTLEAILSFARRG | |
| | | TIG | |

A recombinant process can produce the antigen or RNA, including a linear mRNA, a circular RNA, and both with or without self-replication.

In another embodiment, the polypeptides are native or recombinant peptides and polypeptides derived entirely, partially or mutated from the hypervariable region of the GH loop of the VP1 capsid protein of FMDV (amino acids 135-160) and that contain the RGD motif (sequence of 3 amino acids: Arg-Gly-Asp) described as the sequence that binds integrin receptors of the eukaryotic cell upon infection by FMDV.

### Fusing Carrier Proteins In Vitro

To fuse a sequence of conjugated epitopes of FMD with transferrin, albumin, or Fc via a conceivable peptide bond in vitro, the process involves carefully selecting a conjugation strategy, protein engineering, purification, and ligation. Two primary methods are commonly employed: Sortase-Mediated Ligation (SML) and chemical ligation. In the SML approach, Sortase A, a transpeptidase, recognizes a specific sequence motif (e.g., LPXTG) engineered into transferrin or the target protein and catalyzes the formation of a peptide bond. In chemical ligation, reactive groups, such as cysteine residues, are introduced at specific sites on transferrin and the target protein to enable covalent bonding through thiol-based or other chemistries, such as carbodiimide-mediated amide bond formation or click chemistry.

The process begins with protein engineering, where transferrin and the target protein are modified to include the necessary reactive sites or sequence motifs. For SML, transferrin or the target protein is engineered with an LPXTG motif or a compatible oligo glycine tail. At the same time, chemical ligation requires cysteine residues or functional groups like maleimide or azide-alkyne pairs. These proteins are expressed and purified using systems such as E. coli, yeast, or mammalian cells to ensure proper folding and high purity, often achieved with affinity chromatography. After purification, the ligation reaction is performed. In SML, purified transferrin, the target protein, and Sortase A enzyme are mixed in a buffer such as Tris with calcium ions (if calcium is required by Sortase A) and incubated at 4-37° C. for several hours to promote ligation. In chemical ligation, the proteins are mixed with coupling agents, such as carbodiimides or click chemistry reagents, in a suitable buffer at the optimal pH and temperature to achieve bond formation.

After the ligation reaction, the conjugated product is purified using size-exclusion chromatography or affinity purification techniques to remove unreacted proteins and side products. The success of the conjugation is confirmed through analytical techniques such as SDS-PAGE, Western blot, or mass spectrometry, while functional assays verify the biological activity of the fusion product. Optimization of reaction parameters, including molar ratios, buffer composition, and reaction time, may be necessary to maximize yield and minimize side reactions. Additional stabilization strategies, such as PEGylation, can be considered if the fused product exhibits instability. Combining enzymatic or chemical methods with precise protein engineering allows a stable and biologically active transferrin-protein conjugate with a covalent peptide bond to be successfully synthesized in vitro.

### Genetic Fusion

The genetic fusion begins with the in silico design of the DNA sequence. The open reading frame (ORF) for the protein of interest is joined with the ORF of the ligand (albumin, transferrin, or Fc fragment) through a DNA sequence encoding the non-cleavable peptide linker. Codon optimization is performed to ensure efficient expression in the chosen host system, whether bacterial, yeast, or mammalian cells. For albumin and transferrin, mammalian expression systems are often preferred due to the need for proper folding and post-translational modifications. For Fc fusion proteins, mammalian systems are also ideal to ensure the correct glycosylation and structural stability required for Fc receptor binding and immune interactions.

When choosing a protein-based adjuvant to fuse with a foot-and-mouth disease (FMD) antigen, the goal is to enhance the immunogenicity of the antigen by boosting the innate and adaptive immune responses. Below are protein-based adjuvants that could be used in this context: Flagellin (TLR5 Agonist), C3d (Complement Component), GM-CSF (Granulocyte-Macrophage Colony-Stimulating Factor), PADRE (Pan HLA-DR-binding Epitope), Heat-Shock Proteins (HSPs), and Fc Region of IgG (Table 5).

TABLE 5
Purpose and choice of adjuvants for FMD vaccines
produced by recombinant technology.

| Desired Outcome | Recommended Adjuvant |
|---|---|
| Strong antibody response (humoral) | C3d or Fc region of IgG |
| Balanced Th1/Th2 immunity | Flagellin (TLR5 agonist) |
| Enhanced T-cell response | GM-CSF or PADRE |
| (Th1-biased) | |
| Broad immune activation | Heat-shock proteins (HSPs) |
| (APC targeting) | |
| Mucosal immunity | Cholera Toxin Subunit B (CTB) |

Flagellin and C3d are particularly suitable due to their proven effectiveness in recombinant vaccine platforms.

The construct is inserted into an expression vector with suitable regulatory elements, such as a strong promoter, signal peptide for secretion, and a polyadenylation signal for eukaryotic systems. After transformation into the host cells, the fusion protein is expressed as a single polypeptide chain with the ligand and protein connected by the non-cleavable peptide linker. The expressed fusion protein is then purified using affinity chromatography, exploiting tags like His-tags or natural binding affinities (e.g., Fc binding to Protein A/G or albumin's natural affinity to specific resins).

The resulting fusion protein is characterized to confirm its integrity and activity. Techniques like SDS-PAGE, Western blot, and mass spectrometry are used to verify the fusion protein's molecular weight and sequence accuracy. Functional assays evaluate the biological activity of the ligand and the fused protein, ensuring that the linker does not disrupt functionality. Stability studies confirm that the fusion protein resists cleavage and retains activity under physiological conditions.

Genetic fusion with albumin can enhance the half-life of the protein by leveraging albumin's natural long circulation time. Transferrin fusion can facilitate receptor-mediated delivery into target cells, while Fc fusion can improve therapeutic properties by extending half-life and engaging immune pathways. By employing genetic fusion, stable, biologically active protein-ligand conjugates can be produced in a scalable, cost-effective manner, with applications in therapeutic protein design, drug delivery, and targeted therapies.

### RNA

Linear mRNA Structure: Linear mRNA vaccines are single-stranded RNA molecules engineered to mimic the structure of natural mRNA.

### Basic Structure (Without Self-Replication Components)

5′ Cap: A chemically modified guanosine molecule at the 5′ end, enhancing stability and translation efficiency. Untranslated Regions (UTRs): Sequences flanking the coding region that improve RNA stability and translation. Open Reading Frame (ORF): The coding sequence for the antigenic protein. This is the central part that gets translated into the protein of interest. Poly-A Tail: A string of adenosines at the 3′ end that increases stability and prolongs the mRNA's lifespan in the cytoplasm.

### Enhanced Structure (With Self-Replication Components):

Replicase Genes: Sequences encoding an RNA-dependent RNA polymerase (e.g., derived from alphaviruses like the Semliki Forest virus) are added. These enzymes amplify the mRNA within the cell, allowing for prolonged and enhanced protein production. Subgenomic Promoter: Facilitates transcription of the ORF while keeping replicase separate.

2. Circular RNA (circRNA) Structure

Circular RNA vaccines are covalently closed-loop RNA molecules. They offer increased stability and resistance to exonucleases compared to linear mRNA.

### Basic Structure (Without Self-Replication Components):

Closed Circular Form: The RNA ends are covalently linked, creating a stable loop. Internal Ribosome Entry Site (IRES): A sequence enabling ribosome binding and translation initiation, bypassing the need for a 5′ cap. Coding Region: The antigenic protein-encoding sequence is similar to linear mRNA. UTRs: Help in translation regulation and RNA stability.

### Enhanced Structure (With Self-Replication Components):

Replicase Genes: Like linear mRNA, replicase genes can be included to enable self-replication. Translation Enhancers: Additional elements to boost protein expression, given the unique topology of circRNA.

Delivering a genetically fused protein with albumin, transferrin, or an Fc fragment using linear mRNA or circular RNA (replicating or non-replicating) involves the careful design of RNA constructs that encode the fusion protein, including the ligand, target protein, and a non-cleavable peptide linker. The RNA sequence is engineered to include essential elements for efficient expression. For linear mRNA, these include a 5′ cap for translation initiation, a 5′ untranslated region (UTR) optimized for translational efficiency, an open reading frame (ORF) encoding the fusion protein, and a 3′ UTR and poly(A) tail to ensure stability and translation. For circular RNA, circularization elements such as ribozyme sequences or exonuclease-resistant motifs are included to enable covalent circularization. Codon optimization enhances expression in the host system, such as human cells, while minimizing secondary structures that could impede translation.

An adjuvant is generally unnecessary for an mRNA vaccine encapsulated in lipid nanoparticles (LNPs). LNPs themselves serve as both delivery systems and inherent adjuvants, primarily due to their ability to Enhance Cellular Uptake. LNPs facilitate the delivery of mRNA into cells, enabling the translation of the mRNA into the target antigen. Induce Innate Immune Response: The mRNA within the LNP can stimulate innate immune pathways, such as Toll-like receptors (TLRs) 3, 7, and 8, and other RNA sensors (e.g., RIG-I, MDA5). These innate responses promote the activation of antigen-presenting cells, such as dendritic cells, which are crucial for initiating adaptive immunity. LNPs protect mRNA from enzymatic degradation and facilitate efficient delivery to the cytoplasm. For most applications, an mRNA vaccine with LNPs does not require a separate adjuvant because LNPs already perform multiple functions, including enhancing immunogenicity. However, specific vaccine designs or target populations might occasionally benefit from adjuvant inclusion to tailor or boost immune responses.

mRNA vaccines inherently stimulate TLRs (e.g., TLR3, TLR7, TLR8) due to the presence of single-stranded RNA, inducing innate immunity.

Adding a TLR9-based adjuvant can provide synergistic immune activation, particularly to boost specific immune pathways. CpG Oligonucleotides as TLR9 Agonists: CpG-based adjuvants are compatible with mRNA vaccines and have been used in various vaccine platforms. They can enhance antibody titers and quality, improve the durability of the immune response, and provide a dose-sparing effect, which is crucial for large-scale vaccination efforts. An example of a CpG-based TLR9 agonist is CpG 1018, a synthetic oligodeoxynucleotide (ODN) containing unmethylated CpG motifs. CpG 1018 is widely used as an adjuvant in human and veterinary vaccines. It is one of the most prominent examples of a CpG-based TLR9 agonist approved and successfully used in vaccines. Its demonstrated ability to enhance both humoral and cellular immunity makes it a viable option for designing new vaccines, including those for foot-and-mouth disease (FMD).

Depending on the vaccine's design and immunogenic requirements, flagellin-based adjuvants could be a promising choice for an mRNA vaccine targeting foot-and-mouth disease (FMD).

The construction of non-replicating linear mRNA begins with in vitro transcription (IVT) using a DNA template containing the RNA sequence under a T7 or SP6 promoter. The resulting RNA is capped using methods (e.g., CleanCap) and polyadenylated during transcription or enzymatically post-transcription. The RNA sequence includes viral replicon elements for replicating linear mRNA, such as the non-structural proteins (e.g., nsP1-nsP4) from alphaviruses, enabling intracellular RNA replication. The ORF encoding the fusion protein is placed downstream of a subgenomic promoter within the replicon sequence. The RNA is purified to remove impurities and unwanted by-products, ensuring high-quality RNA for delivery.

Circular RNA is constructed by designing a linear RNA sequence with flanking ribozyme sequences or self-splicing introns, which mediate intramolecular ligation and produce a covalently closed RNA molecule. Alternatively, enzymatic circularization can be performed using RNA ligases. Replicating circular RNA includes additional viral elements that allow intracellular replication, leveraging the stability of the circular structure and prolonging protein expression. Non-replicating and replicating circular RNA are purified to eliminate any remaining linear RNA, ensuring homogeneity of the final product.

The RNA constructs, whether linear or circular, are delivered into cells using lipid nanoparticles (LNPs), which protect the RNA from degradation and facilitate cellular uptake. Alternatively, electroporation or polymeric nanoparticles may be employed depending on the application. Once inside the cells, the host's machinery translates the RNA to produce the genetically fused protein. Linear mRNA offers a more straightforward construction process and is ideal for transient expression, while replicating mRNA provides extended protein expression with lower RNA doses. Circular RNA, on the other hand, is inherently more stable than linear NA due to its resistance to exonucleases, and its replicating variant combines this stability with sustained expression through intracellular replication.

For all RNA types, the ORF encodes the genetically fused protein, starting with the target protein, followed by a non-cleavable peptide linker (e.g., Gly-Ser-Gly-Ser-Gly), and ending with the ligand, such as albumin, transferrin, or an Fc fragment. The resulting fusion protein benefits from the ligand's properties: albumin extends the protein's half-life, transferrin facilitates receptor-mediated delivery, and the Fc fragment enhances therapeutic properties and immune interactions. These RNA platforms are versatile, scalable, and suitable for a wide range of therapeutic and research applications, offering distinct advantages in stability, duration of expression, and ease of design.

Dendrimeric peptides can be expressed using mRNA by designing an mRNA sequence that encodes the desired peptide structure, allowing the host's cellular machinery to synthesize the dendrimeric peptide. These peptides are characterized by a branched structure with a core, often composed of lysine residues, and multiple peptide branches radiating from the core. To achieve this, the mRNA construct includes a coding sequence (ORF) that encodes the dendrimeric peptide, starting with the lysine core and then the functional peptide branches, such as antigenic epitopes from pathogens like FMDV. Flexible or cleavable linkers, such as Gly-Ser-Gly, may be included between branches to ensure proper folding and structural flexibility. The mRNA construct also incorporates essential regulatory elements, including a 5′ cap for translation initiation, a 5′ untranslated region (UTR) optimized for translational efficiency, a 3′ UTR for stabilization, and a poly(A) tail for mRNA stability and prolonged translation. If the dendrimeric peptide is intended for secretion, a signal peptide is included at the beginning of the sequence to direct the peptide to the endoplasmic reticulum (ER). The mRNA is delivered into host cells using lipid nanoparticles (LNPs), which protect the mRNA from degradation and facilitate efficient cellular uptake. Inside the cytoplasm, ribosomes translate the mRNA into the dendrimeric peptide, where the lysine core facilitates the assembly of the branched structure. Proper folding is critical; linkers can help avoid steric hindrance between branches. The dendrimeric peptide may also undergo post-translational modifications if required for functionality, provided the host system supports such processes. This approach enables the scalable and efficient production of dendrimeric peptides for various applications, including vaccines, therapeutics, and diagnostics. For instance, in vaccine development, dendrimeric peptides expressing multiple copies of FMDV epitopes, such as those derived from the VP1 GH loop, can mimic pathogen-like structures, eliciting robust immune responses. However, challenges such as proper folding, the large sequence size of the mRNA, and ensuring immunogenicity without adverse effects must be addressed during design and production. This mRNA-based strategy offers a versatile and cost-effective platform for producing dendrimeric peptides with enhanced stability, bioactivity, and multivalency.

Linear peptides can be expressed using RNA by designing a sequence that encodes the peptide of interest, enabling host cells to synthesize the desired linear peptide efficiently. Unlike dendrimeric peptides, linear peptides have a straightforward structure without branching, making them easier to design and express. The RNA construct is designed to include essential regulatory elements for effective translation, such as a 5′ cap for initiating translation, a 5′ untranslated region (UTR) optimized for efficient ribosome binding, the open reading frame (ORF) encoding the peptide sequence, a 3′ UTR for stability and translational regulation, and a poly(A) tail to enhance mRNA stability and prolong translation. If the peptide requires secretion, a signal peptide is added to the sequence to direct the peptide to the endoplasmic reticulum (ER) for proper processing and secretion.

The coding sequence (ORF) specifically encodes the linear peptide, which may include additional elements like flexible linkers (e.g., Gly-Ser-Gly) to enhance stability or allow for modular design if multiple epitopes or functional domains are included. Codon optimization ensures high translational efficiency in the target host system, whether mammalian or another cell type. RNA is synthesized in vitro using a DNA template under a T7 or SP6 promoter, producing high-quality RNA. This RNA may be linear mRNA, replicating RNA, or circular RNA, depending on the desired duration and yield of peptide expression. Non-replicating linear RNA provides short-term expression, while replicating RNA includes viral replicons that allow intracellular amplification, resulting in sustained peptide production. Circular RNA offers enhanced stability due to resistance to exonucleases and, when replicating elements are added, combines this stability with prolonged antigen expression.

The RNA is delivered into host cells using lipid nanoparticles (LNPs), which protect the RNA from degradation and ensure efficient cellular uptake. Once inside the cytoplasm, the host's ribosomes translate the RNA into the linear peptide. If a signal peptide is included, the peptide is routed through the ER for proper folding, processing, and secretion into the extracellular space. Depending on its intended application, the peptide can act as an immunogen or therapeutic agent. Linear peptides encoded by RNA are suitable for vaccine development, as they can present antigenic epitopes from pathogens like FMDV. For example, a linear RNA encoding peptides derived from the VP1 protein of FMDV, including the hypervariable GH loop region, can elicit a robust immune response, particularly when paired with suitable adjuvants.

Linear peptides expressed via RNA delivery are advantageous due to their simplicity, scalability, and ease of production. They can be tailored to include immunodominant epitopes to enhance immune recognition and are more stable when expressed using advanced RNA delivery methods like LNPs. However, challenges include optimizing RNA stability, translation efficiency, and immunogenicity while avoiding degradation and unwanted inflammatory responses. This approach provides a versatile, cost-effective platform for producing linear peptides for vaccines, therapeutics, and research applications. For instance, combining RNA-delivered linear peptides with adjuvants like CpG ODNs, QS-21, or MPL can significantly enhance the immunogenicity of peptide-based vaccines.

Circular RNA (circRNA) is a versatile platform for delivering linear peptides by encoding the desired peptide sequence within a covalently closed RNA molecule. The circular structure resists exonucleases, making circRNA significantly more stable than linear RNA, and enables prolonged expression of the encoded peptide in the host cells. This inherent stability and ability to sustain peptide production make circRNA an ideal candidate for vaccine applications, therapeutics, and diagnostics. Expressing linear peptides using circRNA involves careful design of the RNA construct to ensure efficient translation and functionality of the peptide.

The circRNA construct includes an open reading frame (ORF) encoding the linear peptide sequence, which may feature immunogenic epitopes, therapeutic sequences, or functional domains tailored to the intended application. The ORF can also incorporate design elements such as signal peptides to direct the linear peptide to the secretory pathway for extracellular release or flexible linkers (e.g., Gly-Ser-Gly) for enhanced stability and modular design. Regulatory elements are integrated to facilitate translation, including internal ribosome entry sites (IRES) or translation-enhancing sequences like optimized UTRs, which allow cap-independent translation in eukaryotic cells. Codon optimization ensures high translational efficiency in the host system, minimizing secondary structures that could impede ribosomal binding and elongation.

To generate circRNA, the RNA is first transcribed as a linear precursor from a DNA template containing self-splicing ribozyme sequences or engineered splice sites at the flanking regions of the ORF. These elements facilitate intramolecular ligation or splicing, creating a covalently closed circular RNA molecule. Alternatively, enzymatic circularization methods, such as RNA ligase-mediated reactions, can generate high-purity circRNA. The circularization process eliminates free RNA ends, protecting the circRNA from exonuclease degradation and providing an extended half-life compared to linear RNA.

The circRNA is delivered into host cells using advanced delivery systems like lipid nanoparticles (LNPs), which protect the RNA from degradation and enhance cellular uptake. Once inside the cytoplasm, the host's translational machinery recognizes the IRES or other translation-initiation elements, allowing efficient production of the linear peptide encoded within the circRNA. If a signal peptide is included in the ORF, the expressed peptide is directed to the endoplasmic reticulum (ER) for proper folding, processing, and secretion into the extracellular space. This process ensures that the peptide functions effectively as an immunogen or therapeutic agent, depending on its intended application.

The use of circRNA for linear peptide delivery offers several advantages, including enhanced stability, prolonged peptide expression, and the ability to achieve sustained immune stimulation or therapeutic effects. For vaccine development, circRNA can encode linear peptides derived from highly immunogenic regions of pathogens, such as the VP1 GH loop of FMDV. This approach enables robust immune responses while reducing the need for frequent dosing. CircRNA is also inherently more stable during storage and transport, making it well-suited for scalable manufacturing and global distribution.

CircRNA delivery faces challenges despite its benefits, including optimizing translation efficiency and ensuring uniform circularization. Advances in RNA engineering, such as improved IRES sequences and high-yield circularization methods, address these limitations and enhance the effectiveness of circRNA-based platforms. Additionally, circRNA can be combined with immunostimulatory adjuvants like CpG ODNs, MPL, or QS-21 to amplify immune responses in vaccine applications. Overall, circular RNA provides a highly stable and efficient platform for delivering linear peptides, offering significant potential in biomedical research and clinical applications.

Self-replicating linear and circular RNA constructs offers an innovative approach for delivering peptides, leveraging their ability to amplify intracellular RNA levels, leading to sustained peptide production over extended periods. These constructs incorporate self-replicating design elements, typically derived from viral replicons, which enable autonomous amplification within the host cells. This strategy significantly enhances the yield of the encoded peptides, reducing the RNA dosage required for adequate therapeutic or immunogenic responses. Both linear and circular self-replicating RNA designs utilize similar principles but differ in structural characteristics and stability.

In self-replicating linear RNA, the construct includes essential replication elements such as non-structural protein (nsP) sequences from RNA viruses like alphaviruses or flaviviruses. These nsP sequences encode RNA-dependent RNA polymerase and accessory proteins that drive replication of the RNA template within the cytoplasm. The RNA construct is designed with a 5′ cap to initiate translation, followed by untranslated regions (UTRs) optimized for stability and replication efficiency. A subgenomic promoter is included downstream of the nsP sequences to drive the expression of the open reading frame (ORF) encoding the target peptide. The ORF can include functional elements such as signal peptides for secretion, flexible linkers, or fusion domains to enhance the stability and activity of the peptide. The 3′ UTR and poly(A) tail are engineered for efficient replication and translational control. Upon delivery into host cells using lipid nanoparticles (LNPs), the self-replicating RNA initiates replication and translation, producing high peptide levels. The transient process provides sustained expression over several days, enabling robust immunogenic or therapeutic effects.

The construct builds on the inherent stability of circular RNA for self-replicating circular RNA while incorporating replicative capabilities. The circular RNA is engineered with viral replication elements, such as replicon sequences from alphaviruses or flaviviruses, that enable intracellular RNA amplification. A ribozyme or enzymatic ligation generates a covalently closed circular structure, eliminating free RNA ends and protecting the molecule from exonucleases. The ORF encoding the peptide is placed downstream of a subgenomic promoter within the replicon, allowing efficient translation of the peptide once replication is initiated. Translation initiation in circular RNA constructs typically relies on internal ribosome entry sites (IRES) or other cap-independent elements, as circular RNA lacks a 5′ cap. The combination of replication-driven amplification and the stability of the circular RNA structure results in prolonged expression of the target peptide, often lasting weeks.

In both linear and circular self-replicating RNA, the target peptide can be designed to include immunogenic epitopes, therapeutic sequences, or diagnostic markers. For example, peptides derived from the VP1 GH loop of FMDV can be encoded in the RNA constructs, providing a strong immunogenic response in vaccine applications. The high yield of peptide production from self-replicating RNA reduces the need for frequent dosing, making it particularly advantageous for scalable vaccine development. Additionally, the constructs can be combined with immunostimulatory adjuvants, such as CpG ODNs, MPL, or QS-21, to enhance the immune response further.

Whether linear or circular, self-replicating RNA systems offer distinct advantages, including high expression levels, prolonged peptide production, and reduced RNA dosages. Linear RNA systems are more straightforward to design and manufacture, while circular RNA systems provide enhanced stability and resistance to degradation. Both systems face challenges, such as ensuring efficient delivery, minimizing off-target effects, and optimizing replication and translation efficiency. Advances in RNA engineering, delivery technologies like LNPs, and integrating stabilizing elements continue to improve the feasibility and effectiveness of self-replicating RNA platforms for peptide delivery. This approach holds immense potential for applications in vaccines, therapeutics, and beyond.

The comparative yield of protein expression from non-replicating linear mRNA, replicating linear mRNA, non-replicating circular RNA, and replicating circular RNA depends on factors such as stability, translation efficiency, and the duration of protein expression. Below is a general comparison of their expected relative yields based on these properties:

The comparative yield of protein expression from non-replicating linear mRNA, replicating linear mRNA, non-replicating circular RNA, and replicating circular RNA depends on factors such as stability, translation efficiency, and the duration of protein expression. Table 6 lists a general comparison of their expected relative yields based on these properties.

TABLE 6
Comparative yield properties and duration of
expression of various RNA delivery systems.

| | | Key Factors | Duration of |
|---|---|---|---|
| RNA Type | Yield | Influencing Yield | Expression |
| Non-Replicating | Moderate | Efficient initial | Short (hours |
| Linear mRNA | | translation due to 5′ | to 1 day) |
| | | cap and optimized | |
| | | UTRs; Susceptible to | |
| | | rapid degradation by | |
| | | nucleases; Limited | |
| | | by the number of | |
| | | RNA molecules | |
| | | delivered. | |
| Replicating | High | Self-replicating RNA | Long |
| Linear mRNA | | amplifies | (several days) |
| | | intracellular RNA | |
| | | levels; Prolonged | |
| | | translation due to | |
| | | continuous RNA | |
| | | production depends | |
| | | on replicon element | |
| | | efficiency. | |
| Non-Replicating | Moderate | High stability due to | Moderate |
| Circular RNA | to High | exonuclease | (1-3 days) |
| | | resistance; Lacks | |
| | | natural cap/poly(A) | |
| | | tail, may require cap- | |
| | | independent | |
| | | translation elements | |
| | | (e.g., IRES). | |
| Replicating | Very High | Combines the | Very Long (up to |
| Circular RNA | | stability of circular | weeks, depending |
| | | RNA with | on the system |
| | | replication-mediated | |
| | | RNA amplification; | |
| | | Highly efficient | |
| | | protein expression | |
| | | over extended | |
| | | periods. | |

The relative quantitative yield of protein expression from the four RNA types can be approximated based on experimental data and known differences in stability, replication, and translation efficiency. While absolute yields depend on experimental conditions, we can provide approximate relative yield ratios to compare their performance (Table 7),

TABLE 7
Relative yield ratios of various RNA forms

| | Relative | |
|---|---|---|
| | Yield | |
| | (Approximate | |
| RNA Type | Ratio) | Explanation |
| Non-Replicating | 1 | Baseline yield with rapid |
| Linear mRNA | | translation but limited by |
| | | mRNA stability and the |
| | | amount delivered. |
| Replicating Linear | 10-50 | Self-replication significantly |
| mRNA | | amplifies RNA levels, |
| | | sustaining high protein |
| | | production over several days. |
| Non-Replicating | 3-5 | Higher stability than linear |
| Circular RNA | | mRNA due to exonuclease |
| | | resistance, leading to |
| | | prolonged translation and |
| | | higher yield. |
| Replicating Circular | 50-100 | Combines stability of circular |
| RNA | | RNA with RNA replication, |
| | | resulting in prolonged and |
| | | exponentially higher yields. |

In one embodiment, the present invention can be designed with any peptide polypeptide or protein as carriers fused to various epitopes derived from FMDV.

The present invention can be designed with any peptide, polypeptide, or protein as carriers fused to various FMDV epitopes in one embodiment. The FMDV epitopes could be entire, partial, or variant sequences of VP1 protein as disclosed in several publications: amino acid residue 144-159 of serotype O1 Kaufbeuren (O1K); amino acid residues 25-41 and 200-213 of serotype O1K; amino acid residue 66-80 of O/UKG/35/2001; amino acid residues 1-12, 17-29 and 194-211 of serotype Asia1.); amino acid residues 106-115 and 4-13 of strain AF/72.

In one embodiment, the peptide or polypeptides are native or recombinant peptides and polypeptides derived entirely, partially, or mutated from VP1 FMDV capsid protein. For example, the peptides derived from VP1 FMDV capsid protein are SEQ ID NO-1-21, as shown in Table 3, and their variants or functional analogs. The hypervariable loop of the VP1 protein among amino acid residue 135-160 is one of the significant sites of phylogenetic diversity between FMDV strains. Percentages of homology for the hypervariable region of the GH loop of the VP1 protein are highly variable. In another embodiment, the polypeptides are native or recombinant peptides and polypeptides derived entirely, partially, or mutated from the hypervariable region of the GH loop of the VP1 capsid protein of FMDV and that contain the RGD motif (sequence of 3 amino acids: Arg-Gly-Asp). Furthermore, peptides derived from FMDV epitopes could be entire, partial, or variant sequences of other capsid proteins, for example, VP2 (amino acid residue 40-50), VP3 (amino acid residue 26-39), and VP4 (amino acid residue 30-41) of serotype Asia1. In addition, the peptides derived from FMDV epitopes could be entire, partial or variant sequences of non-structural proteins (NSP), for example, 2B (PFFFSDVRSNSFKLV (SEQ ID NO.28), FFRSTPEDLERAEK (SEQ ID NO.29)), 2C (LKARDINDIFAILKN (SEQ ID NO.30), SEEKFVTMTDLVPG (SEQ ID NO.31)), 3B (ERTLPGQKACDDVN (SEQ ID NO.35), GPYAGPLETQKPLK (SEQ ID NO.36), PLERQKPLKVRAKL (SEQ ID NO.37), GPYAGPMERQKPLK (SEQ ID NO.38), PMERQKPLKVKAKA (SEQ ID NO.39), QKPLKVKAKAPVVK (SEQ ID NO.40)) from serotype O1K; protein 3A (amino acid residues 11-25 and 21-35), 3C; and protein 3D (amino acid residues 301-315, 326-340, 346-360, 351-365, 356-370 and 406-420) of strain C-S8.

In another embodiment, examples of peptides with FMDV epitopes are shown in Table 3 (SEQ ID NO. 1-55). The present invention also encompasses peptides that are homologous sequences or functional analogs to the peptides of Table 3. In yet another embodiment, the peptides with FMDV epitopes are native or recombinant peptides and polypeptides derived entirely, partially, or mutated from the hypervariable region of the GH loop of the VP1 (135-160) region of FMDV capsid protein and that contain the RGD motif (sequence of 3 amino acids: Arg-Gly-Asp).

The peptide, polypeptide, or protein carriers should be able to present the immunogenic epitopes of the FMDV. In one embodiment, these carriers enhance the immunogenic response. One example of these carriers could be the swine immunoglobulin G heavy-chain constant region that was fused with a tandem-repeat multiple-epitope gene, which contained three copies of each of two immunogens corresponding to amino acid residues 141-160 and 200-213 of VP1 of the FMDV O/China/99 strain. Another example could be the fusion protein designed with the VP1 and bovine IFN-7 sequences that proved to be an inducer of humoral and cell-mediated response. One of the ordinary skills in art is readily recognizing and/or constructing peptide or polypeptide carriers suitable for use in the present invention.

### Adjuvants

The present invention can be designed using different adjuvants, emulsifiers, molecular adjuvants, and carrier systems. In one embodiment, the formulation of the present invention includes, but not limited to, aluminum salts, aluminum hydroxide gel, saponin or derivatives, like QS21, lymph cytokines, CpG, poly I:C, toll-like receptors agonists, immune stimulating complexes (ISCOMs), liposomes, incomplete Freund's adjuvant, liposyn, tyrosine stearate, squalene, L121, Emulsigen, monophosphoryl lipid A (MPL), Montanide ISA adjuvants (ISA 15 VG, ISA 25 VG, ISA 28 VG, ISA 35 VG, ISA 201 VG, ISA 206 VG, ISA 207 VG, ISA 50 V2, ISA 50 V4, ISA 61 VG, ISA 70, ISA 71 VG, ISA 71 R VG, ISA 720, ISA 760, ISA 761 VG, ISA 763 A VG, ISA 775, ISA 780), Montanide IMS adjuvants (IMS 251 C, IMS 1312 VG, IMS 1313 VG N, IMS 2215, IMS 3012), Montanide GEL 01, Montanide GEL 02, light mineral oils, metabolisable oils, polysorbate 20, polysorbate 40, polysorbate 60, polysorbate 65, polysorbate 80, polysorbate 85, polysorbate 120, sorbitan monostearate, sorbitan tristearate, sorbitan monolaurate, sorbitan monooleate, sorbitan trioleate, sorbitan monopalmitate and other efficacious adjuvants and emulsifiers.

In another embodiment, the vaccine formulation is an emulsion, such as a water-in-oil emulsion (W/O), an oil-in-water (O/W) emulsion, or a water-in-oil-in-water emulsion (W/O/W), or an oil-in-water-in-oil (O/W/O) emulsion.

In another embodiment, the vaccine formulation comprises a mix of an emulsion and one or more additional adjuvants. Other examples of carrier systems that could be applied in this vaccine formulation are liposomes that can lead to TH1 or TH2 response, micro/nanospheres, nanoparticles such as poly(lactic-co-glycolic) acid (PLGA) and polysaccharides, dendrimers, micellar systems, gold nanoparticles and Immune-stimulating complexes (ISCOMs) generally known in the art.

In one embodiment, the universal vaccine of the present invention could be administered by syringe injection, needle-free injection, microneedle patch, and delivery. The pharmaceutical combination can be administered by different routes, such as oral, intramuscular (IM), subcutaneous (SC), intradermal (ID), and intranasal spray (INS).

In one embodiment, the pharmaceutical combination of this invention contains antigenic epitopes derived from FMDV capsid protein. The antigenic epitopes may be derived from, for example, A, O, and C serotypes; African SAT1, SAT2, and SAT3 serotypes; and Asia 1 serotype. The antigenic epitope is derived from the FMDV VP1 protein in one embodiment.

### Carrier System Protein as Molecular Adjuvant

In one embodiment, the vaccine formulations of the present invention utilize proteins as carriers of foreign peptides, polypeptides, and/or proteins. In one embodiment, the albumin, transferrin, or Fc is used as a carrier or molecular adjuvant to redirect the immune response towards a specific strain or serotype. In some embodiments, the N-amino end of the carriage protein is fused to a foreign peptide, polypeptides, and/or proteins.

In another embodiment, the foreign peptide, polypeptide, and/or protein comprises FMDV epitopes. In one embodiment, the foreign peptides, polypeptides, and/or proteins are fused to the protein carrier and adjuvant carrier proteins to trigger a strong immune response. In one embodiment, the protein carriers of their variants are fused to FMDV peptides, polypeptides, or proteins derived entirely, partially, or mutated from the capsid proteins VP1, VP2, VP3, and VP4, or non-structural protein 2A, 2B, 2C, 2D, 3A, 3B, 3C and 3D.

In some embodiments, the carrier proteins or their variants are fused to one or more FMDV peptides or their variants or homologs, as shown in Table 3 (SEQ ID NO. 1-55). In another embodiment, the Carrier proteins or their variants are fused to one or more peptides that are homologous sequences or functional analogs to the peptides of Table 1. In one embodiment, the Carrier proteins or their variants are fused to one or more native or recombinant peptides and polypeptides derived entirely, partially, or mutated from the hypervariable region of the GH loop of the VP1 (135-160) region of FMDV capsid protein and that contain the RGD motif (sequence of 3 amino acids: Arg-Gly-Asp) as described above. In another embodiment, the carrier proteins or their variants are fused to proteins, polypeptides, and peptides, with peptides or polypeptides as the linker. In certain embodiments, the fusion proteins can induce an immune response against FMDV in the host.

In various embodiments, the universal vaccine can be formulated using linear peptide epitopes in tandem. In another embodiment, a combination of T and B epitopes can be used

In certain embodiments, in the formulation of the universal vaccine, the polypeptides used to present the immunogenic antigens are dendrimeric peptides that could adopt different configurations. For example, dendrimeric peptides can be configured to contain one or more peptides derived from FMDV epitopes. The FMDV epitopes presented on the dendrimeric peptides could be copies of the same epitope or different epitopes to protect against other strains and/or serotypes of FMDV.

Dendrimeric peptides are highly branched, tree-like molecular structures composed of multiple peptide chains extending from a central core, created using dendrimeric chemistry. These multivalent structures enhance peptides' stability, solubility, and bioactivity, making them particularly useful in immunology, drug delivery, and vaccine development. Their branched architecture allows the presentation of multiple copies of a peptide or different peptides, significantly improving interactions with biological targets. Typical applications include vaccines, where dendrimeric peptides mimic the multivalent antigen presentation of pathogens to elicit strong immune responses, such as displaying FMDV epitopes. In drug delivery, dendrimeric peptides act as carriers, improving solubility and targeted delivery of therapeutics, while in antimicrobial therapy, they amplify the potency of antimicrobial peptides by increasing their selectivity against pathogens. Dendrimeric peptides are also valuable in cancer therapy and molecular imaging, enabling targeted drug delivery or diagnostic tagging. Their advantages include multivalency, enhanced immunogenicity, resistance to proteolytic degradation, and customizable structures for specific applications. Platforms like multiple antigen peptides (MAPs) and polylysine dendrimers exemplify their utility. However, challenges such as synthesis complexity, potential size-related limitations, and cytotoxicity require careful design. Dendrimeric peptides represent a versatile and promising platform for advancing vaccines, therapeutics, and diagnostics. In another embodiment, the dendrimeric peptides can only be constructed by fusing FMDV immunogenic peptides.

In one embodiment, the universal vaccine formulation comprises one or more carrier proteins, chimeric proteins carrying different peptides, polypeptides, and/or proteins derived from different FMDV types to give protection against various kinds of FMDV.

In another embodiment, different peptides, polypeptides, and/or proteins from FMDV can be fused to the carrier protein-protein to protect against various types of FMDV.

In certain embodiments, one or more peptides, polypeptides, and/or proteins from FMDV fused to the carrier proteins protein derived from B epitopes and/or T epitopes of FMDV.

In one embodiment, the criteria for choosing different peptides, polypeptides, and/or proteins from FMDV to be fused with carrier proteins depend on the type of FMDV one needs protection against. This invention can design vaccines against one specific strain, against different strains of the same serotype, or other serotypes. For protection against a particular strain, a combination of peptides derived from the B and T epitope of that strain is recommended to reach a high protection level. For protection against more than one strain, it is necessary to combine peptides derived from epitopes of different strains, preferably peptides derived from both B epitope and T epitope of FMDV. For protection against more than one serotype, it is necessary to combine peptides derived from epitopes of different serotypes, preferably peptides derived from both B epitope and T epitope.

In one embodiment, examples of peptides with FMDV epitopes include but are not limited to, the peptides described in Table 3. One would recognize that the present invention is not limited to the following peptides: it encompasses the following peptides and their variants, homologous sequences, and/or functional analogs.

In one embodiment, the present invention is suitable to qualify as an emergency vaccine under the OIE protocol to be used against outbreaks of emerging FMDV strains. This qualification is achieved because this vaccine provides animals with sufficient protection against FMDV infection after a single administration.

In one embodiment, the present invention is suitable to generate antigen banks that could be used in an emergency to formulate an FMDV universal vaccine.

In another embodiment, the vaccine formulations can be administered in multiple doses.

In one embodiment, the present invention provides a vaccine formulation capable of inducing cross-protection against different serotypes and/or strains of Foot and Mouth Disease Virus (FMDV), comprising encoding FMDV peptides, polypeptides or proteins in various types of plasmids; b) recombinant FMDV peptides, polypeptides or proteins; c) peptides, polypeptides or proteins used as a carrier or as molecular adjuvant fused to peptides, polypeptides and/or proteins derived from FMDV; d) adjuvants; emulsifiers, molecular adjuvants and carrier systems.

In one embodiment, the above vaccine formulation can induce protective immunity against all strains of a given serotype of FMDV. In another embodiment, the vaccine formulation can induce protective immunity against all strains of one or more serotypes: O, A, C, Asia 1, SAT-1, SAT-2, and SAT-3. In another embodiment, the vaccine formulation can induce protective immunity against all strains of all serotypes of FMDV.

In one embodiment, the vaccine formulation comprises one or more polynucleotides that encode the entire or partial or variant of FMDV proteins, such as capsid protein genes VP1, VP2, VP3 and VP4; non-structural protein genes 2A, 2B, 2C, 2D, 3A, 3B, 3C and 3D; or any polynucleotide sequences that encode one of the peptides of SEQ ID NO. 1-55 (Table 3) or its variants. In another embodiment, the vaccine formulation comprises polynucleotides that encode peptide(s) homologous to the peptides of Table 1. In yet another embodiment, the vaccine formulation comprises polynucleotides that encode peptide(s) that are functional analogs to the peptides of Table 3.

In one embodiment, the vaccine formulation comprises FMDV peptides, polypeptides, or proteins comprising the entire or partial or variant sequences of one or more FMDV proteins such as capsid proteins VP1, VP2, VP3, and VP4; non-structural proteins 2A, 2B, 2C, 2D, 3A, 3B, 3C and 3D; or peptides of SEQ ID NO. 1-55. In another embodiment, the polypeptides or proteins comprise any amino acid sequences that are homologous or functional analogs to the peptides of Table 1. In yet another embodiment, the FMDV polypeptides are native or recombinant peptides and polypeptides derived entirely, partially, or mutated from the hypervariable region of the GH loop of the VP1 (135-160) region of FMDV capsid protein and that contain the RGD motif (sequence of 3 amino acids: Arg-Gly-Asp). In one embodiment, the FMDV polypeptides are linear peptides. In another embodiment, the FMDV polypeptides are dendrimeric peptides with different configurations, including but not limited to random hyperbranched, dendrigraft, dendrons, and dendrimers. The dendrimeric peptides are only constructed by fusing FMDV immunogenic peptides in one embodiment.

In one embodiment, the inactivated FMDV used in the vaccine formulation can originate from any serotype or strain of FMDV. In another embodiment, the vaccine formulation comprises one or more inactivated FMDV originating from different serotypes and/or strains of FMDV.

In one embodiment, the vaccine formulation comprises peptides, polypeptides, and proteins, used as carriers and/or molecular adjuvants, that are fused to entire or partial or variant amino acid sequences of one or more FMDV peptides, polypeptides and proteins, such as capsid proteins VP1, VP2, VP3 and VP4; non-structural proteins 2A, 2B, 2C, 2D, 3A, 3B, 3C and 3D; peptides of SEQ ID NO. 1-55, or any homologous or functional peptides analogous to the peptides of Table 1. In one embodiment, the FMDV polypeptides are native, or recombinant peptides and polypeptides derived entirely, partially, or mutated from the hypervariable region of the GH loop of the VP1 (135-160) region of FMDV capsid protein as described above.

The protein, polypeptide, and peptide carriers can be fused to the target sequences with any linker in one embodiment.

In another embodiment, the carrier proteins are fused to peptides, polypeptides, and proteins with any peptide or polypeptide linker. In one embodiment, the carrier proteins are fused to one or more FMDV peptides, polypeptides, and proteins from the same FMDV strain or serotype. In another embodiment, the carrier proteins are fused to one or more FMDV peptides, polypeptides, and proteins from different FMDV strains and/or serotypes. In another embodiment, the carrier proteins or their variants are not fused to any FMDV peptides, polypeptides, and proteins. In another embodiment, variants of carrier proteins are carrier proteins with point mutations that improve the degree of stability.

In one embodiment, the vaccine formulation is an emulsion, such as a water-in-oil emulsion (W/O), an oil-in-water (O/W) emulsion, or a water-in-oil-in water emulsion (W/O/W), or an oil-in-water-in oil (O/W/O) emulsion. In another embodiment, the vaccine formulation comprises a mix of an emulsion and one or more additional adjuvants.

The vaccine formulation comprises a lipid nanoparticle (LNP) in one embodiment.

In one embodiment, the carrier systems can be liposomes, microspheres, nanoparticles, dendrimers, micellar systems, or immune stimulating complexes (ISCOMs).

The present invention also provides a method of vaccinating a host susceptible to FMDV infection, comprising administrating the vaccine formulation described above to the host to induce an immune response.

The host has not been infected with FMDV in one embodiment, and the induced immune response is protective. In another embodiment, the host has been infected with FMDV, and the induced immune response is therapeutic. In one embodiment, the induced immune response is the humoral immune response. In another embodiment, the immune response is a cellular immune response. In another embodiment, the induced immune response comprises cross-protective neutralizing antibodies against various serotypes and/or strains of FMDV. In yet another embodiment, the induced immune response cross-reacts against various serotypes and/or strains of FMDV.

In one embodiment, the vaccine formulation can induce protective immunity against all strains of a given serotype of said virus or against all strains of all serotypes of said virus.

In one embodiment, the vaccine formulation is against FMDV. The polynucleotides in the formulation are derived from the entire, partial, or variant sequences of: (a) polynucleotide sequences encoding one or more of FMDV capsid protein genes VP1, VP2, VP3 and VP4; (b) polynucleotide sequences encoding one or more of FMDV non-structural protein genes 2A, 2B, 2C, 2D, 3A, 3B, 3C and 3D; (c) polynucleotide sequences encoding one or more of peptides of SEQ ID NO. 1-55; or (d) polynucleotide sequences encoding peptides that are homologous to the peptides of Table 3.

In another embodiment, the vaccine formulation is against FMDV. The recombinant or viral peptides, polypeptides, or proteins are encoded by the entire, partial, or variant sequences of: (a) amino acid sequences of FMDV capsid proteins VP1, VP2, VP3 and VP4; (b) amino acid sequences of FMDV non-structural proteins 2A, 2B, 2C, 2D, 3A, 3B, 3C and 3D; (c) amino acid sequences of one or more of SEQ ID NO. 1-55; or (d) amino acid sequences that encode peptides that are homologous or functional analogs to the peptides of Table 3.

The recombinant or viral peptides or polypeptides are linear or dendrimeric in one embodiment. In another embodiment, the vaccine formulation comprises inactivated FMDV derived from any serotype or strain of FMDV. In one embodiment, the inactivated FMDV shall consist of one or more serotypes or strains of FMDV.

In another embodiment, the vaccine formulation is against FMDV. The carriers or molecular adjuvants are fused to entire, partial, or variant of: (a) amino acid sequences of FMDV capsid proteins VP1, VP2, VP3, and VP4; (b) amino acid sequences of FMDV non-structural proteins 2A, 2B, 2C, 2D, 3A, 3B, 3C and 3D; (c) amino acid sequences of one or more of SEQ ID NO. 1-55; or (d) amino acid sequences that are homologous or functional analogs to the peptides of Table 3. In one embodiment, homologous peptides may have 55, 60, 65, 70, 75, 80, 85, 90, or 95% homology to SEQ ID NO.1-55. In another embodiment, functional analogous peptides may only have as low as 10% amino acid sequence homology to any one of SEQ ID NO.1-55. In one embodiment, the carriers or molecular adjuvants are linear or dendrimeric.

In another embodiment, the carrier's proteins molecular adjuvants are derived from a native amino acid sequence of serum proteins of cattle.

The present invention also provides a method of vaccinating a host susceptible to FMDV infection, comprising administrating the vaccine formulation described above to the host to induce an immune response. In some embodiments, the vaccine formulation components are administrated simultaneously but in different locations of the host. In certain embodiments, the components of the vaccine formulation are administrated at different time points in the same location as the host. In one embodiment, the components of the vaccine formulation are administrated at different time points in other places of the host. The host is a cattle, sheep, goats or swine.

The host has not been infected with FMDV in one embodiment, and the induced immune response is protective. In another embodiment, the induced immune response is the humoral or cellular immune response.

In one embodiment, the induced immune response comprises cross-protective neutralizing antibodies against various serotypes or strains of FMDV. In another embodiment, the induced immune response cross-reacts against various serotypes or strains of FMDV.

In some embodiments, the vaccine formulation used in the pharmaceutical combination of the present invention can induce cross-protection against different serotypes and/or strains of a target virus FMDV.

In one embodiment, the carrier protein-protein is fused to the components that can trigger immune responses by peptide or polypeptide linkers.

In one embodiment, the pharmaceutical combination can be administered simultaneously, along with the exact body location of the host. In another embodiment, the pharmaceutical combination can be administered at different times and the precise body location of the host. In another embodiment, the pharmaceutical combination can be administered simultaneously and at various body locations of the host. In another embodiment, the pharmaceutical combination can be administered at different times and other body locations of the host.

1. A fusion universal vaccine formulation capable of inducing cross-protection against different serotypes or strains of Food and Mouth Disease virus (FMDV), comprising recombinant or RNA-delivered peptides, polypeptides, or proteins of known sera of the said FMDV fused with serum proteins to extend the half-life of the antigen.

2. The fusion universal vaccine formulation of claim 1, wherein the peptides, polypeptides, or proteins are constructed from epitopes of O, A, C, Asia 1, SAT-1, SAT-2, and SAT-3 sera, or a combination thereof.

3. The fusion universal vaccine formulation of claim 1, wherein the formulation can induce protective immunity against one or more serotypes or strains selected from the group consisting of O, A, C, Asia 1, SAT-1, SAT-2, and SAT-3 or a combination thereof.

4. The vaccine formulation of claim 2, wherein said peptides, polypeptides, or proteins are derived from the same or different FMDV strain(s) or serotype(s).

5. The vaccine formulation of claim 4, wherein the epitopes of FMDV are derived from genes comprising AY593763, EF208770, EF208771, EF208772, EF208769, FJ755007, FJ755133, FJ755155, FJ755010, KY091291, KY091294, KY091290, KY091293, KY091296, KY091295, KY091297, AY593755, EF208777, EF208778, HQ116312, AF390646, KF561699, EF208756, AY593761, GU566064, KF561704, KF561698, KF561705, M10975, AY593780, AY593768, AJ306219, AY593766, EU553852, AY593795, DQ121109, DQ121119, DQ101240, FJ785230, DQ121116, AF390678, KY091304, JF739177, AY304994, AY390432, KY091300, FJ798151, KY091302, KY091301, M90381, AJ133357, M90376, KY091303, EU553893, AY593810, AJ294911, AJ294919, AJ294926, AF308157, AJ303500, AJ303502, AJ303503, AJ303509, AJ303501, AY593815, M55287, AJ004645, DQ834727, AJ303511, KY091280, AJ296327, DQ165074, DQ165077, DQ165073, KF561679, DQ165075, FJ798109, FJ798127, FJ798137, HM211075, FJ798141, AJ303488, AJ303485, AY593823, AF204276, AF292107, AJ539141, EU667451, KY091281, KY091284, KY091283, KY091282, KY091285, DQ164904, DQ164941, KM921876, KM921814, KY091288, AJ294910, DQ164925, KF561706, KF219690, AY593839, AY593846, AY593838, AY593845, KF219682, AF431711, AY593844, DQ009725, AY442010, AY442012, FJ798154, KX822796, KF219691, AY593848, KF219689, AF136607, EF134951, AY593847, AF367124, AY344505, AY343935, AF367139, AF479415, AF479410, AF367135, JX570616, DQ009737, AF367134, AJ251473, AY343941, AF367100, AY343969, AF479417, AY343963, AY343939, FJ798161, and AY343938, or a combination thereof.

6. The fusion universal vaccine formulation of claim 1, wherein the serum proteins comprise Albumin (BSA), IgG2 (Fc gamma), IgG1, Transferrin, Complement Factor H, Fibrinogen, Thyroglobulin, Factor IX, C-Reactive Protein, Retinol-Binding Protein (RBP), Transcription Factor VIIa, G-CSF, and Interferon-Alpha (IFN-α), or a combination thereof.

7. The fusion universal vaccine formulation of claim 6, wherein the preferred serum proteins comprise albumin, transferrin, and Fc region, or a combination thereof.

8. The fusion universal vaccine formulation of claim 1, wherein the antigen is presented by Sequence No 67.

9. The fusion universal vaccine formulation of claim 1, wherein the fused vaccine antigen with short peptides, is presented by Sequence No 70.

10. The fusion universal vaccine formulation of claim 7, wherein short peptides capable of binding albumin, Fc, and transferrin, or a combination thereof, replace albumin, Fc, and transferrin.

11. The fusion universal vaccine of claim 1, wherein the serum proteins are fused in vitro by a chemical reaction, expressed as a combined protein using recombinant technology or delivered using linear mRNA, circular RNA, both with and without self-replication.

12. The fusion universal vaccine formulation of claim 1, wherein an adjuvant is fused with the antigenic peptide, polypeptide, or protein when the vaccine is produced using recombinant technology.

13. The fusion universal vaccine formulation of claim 10, wherein adjuvant comprises Alpha-Gal-Based Adjuvants, Alpha-Galactosylceramide (α-GalCer), Beta-Glucans, C3d (Complement Component), CAF01 (Cationic Liposomes), Calcium Phosphate Nanoparticles, Chitosan (for mucosal vaccines), Chitosan Microparticles, Cholera Toxin B Subunit (CTB), CpG ODN (TLR9 agonist), Cyclic Dinucleotides (e.g., c-di-GMP, c-di-AMP), Cytosine-Phosphate-Guanine Dinucleotides (CpG ODNs), Defensins (Other than hBD-3), Dendritic Cell-Targeting Antibodies, Flagellin (TLR5 agonist), Flagellin Variants (e.g., FliC), Flt3 Ligand, GM-CSF (Cytokine), Heat Shock Protein 70 (HSP70), Heat-Inactivated Bacteria, Human β-Defensin 3 (hBD-3), Hyaluronic Acid Derivatives, IL-12 (Cytokine), Imiquimod (TLR7 agonist), Immunostimulatory Complex (ISCOMATRIX), Interferon-α (IFN-α), ISCOMs (Immune-Stimulating Complexes), Leukotoxin Derivatives, MF59 (Oil-in-Water Emulsion), Microneedle Patches, Monophosphoryl Lipid A (MPL), Muramyl Dipeptide (MDP), Nanodiamonds, PADRE (Pan HLA-DR Epitope), Peptidoglycan Fragments, Plant-derived saponins (e.g., Ginseng Saponins), PLGA Nanoparticles, Poly(I:C) (TLR3 agonist), Polymer-based adjuvants (e.g., Polyethyleneimine, PEI), Polyphosphazene (e.g., PCEP), QS-21 (Saponin-based), R848 (TLR7/8 agonist), Resiquimod (R848, TLR7/8 agonist), Retinoic Acid (RA), Saponin-Based Derivatives (e.g., Quil-A), Squalene-Based Emulsions, STING Agonists (e.g., cGAMP), Synthetic Nanoadjuvants (e.g., Toll-Like Receptor Agonist-Conjugated Nanoparticles), Synthetic RNA Sensors (e.g., RIG-I Agonists), Tetanus Toxin Fragment C (TTFc), TLR Ligand Mixtures, TLR2 Agonists (e.g., Pam3CSK4), TLR4 Agonists (e.g., GLA-SE), Toll-Like Receptor 3 Agonists (e.g., Polyinosinic: polycytidylic acid, Poly(I:C)), Trehalose Dibehenate (TDB), Trehalose-6,6-dimycolate (TDM), Uric Acid Crystals, Virosomes (Lipid Bilayer Vesicles with Viral Components), and Zymosan (TLR2/6 agonist) or a combination thereof.

14. The fusion universal vaccine of claim 11, wherein the preferred adjuvant is Flagellin (TLR5 Agonist) (Sequence 61).

15. The vaccine formulation of claim 1, wherein said recombinant or RNA-delivered peptides, polypeptides, or proteins are encoded by the entire, partial, or variant sequences of:
a. amino acid sequences of FMDV capsid proteins VP1, VP2, VP3 and VP4;
b. amino acid sequences of FMDV non-structural proteins 2A, 2B, 2C, 2D, 3A, 3B, 3C and 3D;
c. amino acid sequences of one or more of SEQ ID NO. 1-55; or
d. amino acid sequences encoding peptides that are homologous or functional analogs to one or more of SEQ ID NO. 1-55.

16. The vaccine formulation of claim 1, wherein said recombinant or RNA-delivered peptides or polypeptides are linear or dendrimeric peptides.

17. A method of vaccinating a host susceptible to FMDV infection, comprising administrating one or more of the vaccine formulations of claim 1 to induce an immune response.

18. The method of claim 1, wherein the host is a cow, sheep, goat, or swine.

19. The method of claim 1, wherein the induced immune response is a humoral or cellular immune response.

20. The method of claim 1, wherein the induced immune response comprises cross-protective neutralizing antibodies against two or more serotypes or strains of FMDV.


SEQUENCE LISTING (71 sequences)

SEQ ID NO: 1 - protein, 12 aa, Foot-and-mouth disease virus
TTTTGESADPVT

SEQ ID NO: 2 - protein, 10 aa, Foot-and-mouth disease virus
TGESADPVTT

SEQ ID NO: 3 - protein, 13 aa, Foot-and-mouth disease virus
NYGGETQTARRLH

SEQ ID NO: 4 - protein, 20 aa, Foot-and-mouth disease virus
ETQIQRRQHTDVSFIMDRFV

SEQ ID NO: 5 - protein, 17 aa, Foot-and-mouth disease virus
QRRQHTDVSFIMDRFVK

SEQ ID NO: 6 - protein, 9 aa, Foot-and-mouth disease virus
RRQHTDVSF

SEQ ID NO: 7 - protein, 15 aa, Foot-and-mouth disease virus
LRTATYYFADLEVAV

SEQ ID NO: 8 - protein, 10 aa, Foot-and-mouth disease virus
AYHKGPFTRL

SEQ ID NO: 9 - protein, 21 aa, Foot-and-mouth disease virus
YSRNAVPNARGDLQVLAQKVA

SEQ ID NO: 10 - protein, 21 aa, Foot-and-mouth disease virus
YTVSGLSRRGDLGSLAARVAK

SEQ ID NO: 11 - protein, 21 aa, Foot-and-mouth disease virus
YTGGSLPNVRGDLQVLAPKAA

SEQ ID NO: 12 - protein, 21 aa, Foot-and-mouth disease virus
YGESDVTNVRGDLQVLAQKAA

SEQ ID NO: 13 - protein, 21 aa, Foot-and-mouth disease virus
YGENNVTNVRGDLQVLAQKAA

SEQ ID NO: 14 - protein, 21 aa, Foot-and-mouth disease virus
SKYSAPQNRRGDLGPLAARLA

SEQ ID NO: 15 - protein, 21 aa, Foot-and-mouth disease virus
SKYSTPQTRRGDLGPLAARLA

SEQ ID NO: 16 - protein, 21 aa, Foot-and-mouth disease virus
AVPNARGDLQVLAQKVARTLP

SEQ ID NO: 17 - protein, 21 aa, Foot-and-mouth disease virus
SLPNVRGDLQVLAPKAARPLP

SEQ ID NO: 18 - protein, 16 aa, Foot-and-mouth disease virus
LRGDLQVLAQKVARTL

SEQ ID NO: 19 - protein, 9 aa, Foot-and-mouth disease virus
RTLPTSFNY

SEQ ID NO: 20 - protein, 18 aa, Foot-and-mouth disease virus
TTQDRRKQEIIAPEKQTL

SEQ ID NO: 21 - protein, 13 aa, Foot-and-mouth disease virus
HKQKIVAPVKQTL

SEQ ID NO: 22 - protein, 11 aa, Foot-and-mouth disease virus
EDAVSGPNTSG

SEQ ID NO: 23 - protein, 15 aa, Foot-and-mouth disease virus
PFGHLTKLELPTDHH

SEQ ID NO: 24 - protein, 14 aa, Foot-and-mouth disease virus
YGKVSNPPRTSFPG

SEQ ID NO: 25 - protein, 15 aa, Foot-and-mouth disease virus
DVSLAAKHMSNTYLS

SEQ ID NO: 26 - protein, 16 aa, Foot-and-mouth disease virus
SIINNYYMQQYQNSMD

SEQ ID NO: 27 - protein, 12 aa, Foot-and-mouth disease virus
YQNSMDTQLGDN

SEQ ID NO: 28 - protein, 15 aa, Foot-and-mouth disease virus
PFFFSDVRSNFSKLV

SEQ ID NO: 29 - protein, 14 aa, Foot-and-mouth disease virus
FFRSTPEDLERAEK

SEQ ID NO: 30 - protein, 15 aa, Foot-and-mouth disease virus
LKARDINDIFAILKN

SEQ ID NO: 31 - protein, 14 aa, Foot-and-mouth disease virus
SEEKFVTMTDLVPG

SEQ ID NO: 32 - protein, 15 aa, Foot-and-mouth disease virus
VTMTDLVPGILEKQR

SEQ ID NO: 33 - protein, 15 aa, Foot-and-mouth disease virus
YFLIEKGQHEAAIEF

SEQ ID NO: 34 - protein, 15 aa, Foot-and-mouth disease virus
AAIEFFEGMVHDSIK

SEQ ID NO: 35 - protein, 14 aa, Foot-and-mouth disease virus
ERTLPGQKACDDVN

SEQ ID NO: 36 - protein, 14 aa, Foot-and-mouth disease virus
GPYAGPLETQKPLK

SEQ ID NO: 37 - protein, 14 aa, Foot-and-mouth disease virus
PLERQKPLKVRAKL

SEQ ID NO: 38 - protein, 14 aa, Foot-and-mouth disease virus
GPYAGPMERQKPLK

SEQ ID NO: 39 - protein, 14 aa, Foot-and-mouth disease virus
PMERQKPLKVKAKA

SEQ ID NO: 40 - protein, 14 aa, Foot-and-mouth disease virus
QKPLKVKAKAPVVK

SEQ ID NO: 41 - protein, 14 aa, Foot-and-mouth disease virus
PVKKPVALKVKAKN

SEQ ID NO: 42 - protein, 15 aa, Foot-and-mouth disease virus
NADVGRLIFSGEALT

SEQ ID NO: 43 - protein, 15 aa, Foot-and-mouth disease virus
AVLAKDGADTFIVGT

SEQ ID NO: 44 - protein, 15 aa, Foot-and-mouth disease virus
MRKTKLAPTVAHGVF

SEQ ID NO: 45 - protein, 15 aa, Foot-and-mouth disease virus
VLDEVIFSKHKGDTK

SEQ ID NO: 46 - protein, 23 aa, Foot-and-mouth disease virus
TANAPLSIYEAIKGVDGLDAMEP

SEQ ID NO: 47 - protein, 20 aa, Foot-and-mouth disease virus
VDVLPVEHILYTRMMIGRFC

SEQ ID NO: 48 - protein, 15 aa, Foot-and-mouth disease virus
SATSIINTILNNIYV

SEQ ID NO: 49 - protein, 15 aa, Foot-and-mouth disease virus
VELDTYTMISYGDDI

SEQ ID NO: 50 - protein, 20 aa, Foot-and-mouth disease virus
VVASDYDLDFEALKPHFKSL

SEQ ID NO: 51 - protein, 15 aa, Foot-and-mouth disease virus
YDLDFEALKPHFKSL

SEQ ID NO: 52 - protein, 15 aa, Foot-and-mouth disease virus
EALKPHFKSLGQTYT

SEQ ID NO: 53 - protein, 15 aa, Foot-and-mouth disease virus
HFKSLGQTYTPADKS

SEQ ID NO: 54 - protein, 20 aa, Foot-and-mouth disease virus
TDVTFLKRHFHMDYGTGFYK

SEQ ID NO: 55 - protein, 15 aa, Foot-and-mouth disease virus
KTLEAILSFARRGTI

SEQ ID NO: 56 - protein, 15 aa, synthetic construct
GGGGSGGGGSGGGGS

SEQ ID NO: 57 - protein, 1679 aa, Foot-and-mouth disease virus
TTTTGESADPVTGGGGSGGGGSGGGGSTGESADPVTTGGGGSGGGGSGGGGSNYGGETQTARRLHGGGGSGGGGSGGGGSETQIQRRQHTDVSFIMDRFVGGGGSGGGGSGGGGSQRRQHTDVSFIMDRFVKGGGGSGGGGSGGGGSRRQHTDVSFGGGGSGGGGSGGGGSLRTATYYFADLEVAVGGGGSGGGGSGGGGSAYHKGPFTRLGGGGSGGGGSGGGGSYSRNAVPNARGDLQVLAQKVAGGGGSGGGGSGGGGSYTVSGLSRRGDLGSLAARVAKGGGGSGGGGSGGGGSYTGGSLPNVRGDLQVLAPKAAGGGGSGGGGSGGGGSYGESDVTNVRGDLQVLAQKAAGGGGSGGGGSGGGGSYGENNVTNVRGDLQVLAQKAAGGGGSGGGGSGGGGSSKYSAPQNRRGDLGPLAARLAGGGGSGGGGSGGGGSSKYSTPQTRRGDLGPLAARLAGGGGSGGGGSGGGGSAVPNARGDLQVLAQKVARTLPGGGGSGGGGSGGGGSSLPNVRGDLQVLAPKAARPLPGGGGSGGGGSGGGGSLRGDLQVLAQKVARTLGGGGSGGGGSGGGGSRTLPTSFNYGGGGSGGGGSGGGGSTTQDRRKQEIIAPEKQTLGGGGSGGGGSGGGGSHKQKIVAPVKQTLGGGGSGGGGSGGGGSEDAVSGPNTSGGGGGSGGGGSGGGGSPFGHLTKLELPTDHHGGGGSGGGGSGGGGSYGKVSNPPRTSFPGGGGGSGGGGSGGGGSDVSLAAKHMSNTYLSGGGGSGGGGSGGGGSSIINNYYMQQYQNSMDGGGGSGGGGSGGGGSYQNSMDTQLGDNGGGGSGGGGSGGGGSPFFFSDVRSNFSKLVGGGGSGGGGSGGGGSFFRSTPEDLERAEKGGGGSGGGGSGGGGSLKARDINDIFAILKNGGGGSGGGGSGGGGSSEEKFVTMTDLVPGGGGGSGGGGSGGGGSVTMTDLVPGILEKQRGGGGSGGGGSGGGGSYFLIEKGQHEAAIEFGGGGSGGGGSGGGGSAAIEFFEGMVHDSIKGGGGSGGGGSGGGGSERTLPGQKACDDVNGGGGSGGGGSGGGGSGPYAGPLETQKPLKGGGGSGGGGSGGGGSPLERQKPLKVRAKLGGGGSGGGGSGGGGSGPYAGPMERQKPLKGGGGSGGGGSGGGGSPMERQKPLKVKAKAGGGGSGGGGSGGGGSQKPLKVKAKAPVVKGGGGSGGGGSGGGGSPVKKPVALKVKAKNGGGGSGGGGSGGGGSNADVGRLIFSGEALTGGGGSGGGGSGGGGSAVLAKDGADTFIVGTGGGGSGGGGSGGGGSMRKTKLAPTVAHGVFGGGGSGGGGSGGGGSVLDEVIFSKHKGDTKGGGGSGGGGSGGGGSTANAPLSIYEAIKGVDGLDAMEPGGGGSGGGGSGGGGSVDVLPVEHILYTRMMIGRFCGGGGSGGGGSGGGGSSATSIINTILNNIYVGGGGSGGGGSGGGGSVELDTYTMISYGDDIGGGGSGGGGSGGGGSVVASDYDLDFEALKPHFKSLGGGGSGGGGSGGGGSYDLDFEALKPHFKSLGGGGSGGGGSGGGGSEALKPHFKSLGQTYTGGGGSGGGGSGGGGSHFKSLGQTYTPADKSGGGGSGGGGSGGGGSTDVTFLKRHFHMDYGTGFYKGGGGSGGGGSGGGGSKTLEAILSFARRGTIG

SEQ ID NO: 58 - protein, 448 aa, Bos taurus
MEQLRESGPSLVKPSQTLSLTCTVSGFSLNDNRVVWVRQPPGKAPEWLGRIYSGGATHINPALKSRLSISKDNSKNEVSLSISDVRTEDTATYMCGKEQTFNDGESSYIDVWGQGFRVIVSSASTTAPKVYPLTSCCGDKSSSRVTLGCLVSSYMPEPVTVTWNSGALKSGVHTFPAVLQSSGLYSLSSMVTVPGSSSGQTFTCNVAHPASSTKVDKAVGVSSDCSKPNNQHCVREPSVFIFPPKPKDTLMITGTPEVTCVVVNVGHDNPEVQFSWFVDDVEVHTARTKPREEQFNSTYRVVSALPIQHQDWTGGKEFKCKVNIKGLSASIVRIISRSKGPAREPQVYVLDPPKEELSKSTVSLTCMVIGFYPEDVDVEWQRDRQTESEDKYRTTPPQLDADRSYFLYSKLRVDRNSWQRGDTYTCVVMHEALHNHYMQKSTSKSAGK

SEQ ID NO: 59 - protein, 607 aa, Bos taurus
MKWVTFISLLLLFSSAYSRGVFRRDTHKSEIAHRFKDLGEEHFKGLVLIAFSQYLQQCPFDEHVKLVNELTEFAKTCVADESHAGCEKSLHTLFGDELCKVASLRETYGDMADCCEKQEPERNECFLSHKDDSPDLPKLKPDPNTLCDEFKADEKKFWGKYLYEIARRHPYFYAPELLYYANKYNGVFQECCQAEDKGACLLPKIETMREKVLASSARQRLRCASIQKFGERALKAWSVARLSQKFPKAEFVEVTKLVTDLTKVHKECCHGDLLECADDRADLAKYICDNQDTISSKLKECCDKPLLEKSHCIAEVEKDAIPENLPPLTADFAEDKDVCKNYQEAKDAFLGSFLYEYSRRHPEYAVSVLLRLAKEYEATLEECCAKDDPHACYSTVFDKLKHLVDEPQNLIKQNCDQFEKLGEYGFQNALIVRYTRKVPQVSTPTLVEVSRSLGKVGTRCCTKPESERMPCTEDYLSLILNRLCVLHEKTPVSEKVTKCCTESLVNRRPCFSALTPDETYVPKAFDEKLFTFHADICTLPDTEKQIKKQTALVELLKHKPKATEEQLKTVMENFVAFVDKCCAADDKEACFAVEGPKLVVSTQTALA

SEQ ID NO: 60 - protein, 704 aa, Bos taurus
MRPAVRALLACAVLGLCLADPERTVRWCTISTHEANKCASFRENVLRILESGPFVSCVKKTSHMDCIKAISNNEADAVTLDGGLVYEAGLKPNNLKPVVAEFHGTKDNPQTHYYAVAVVKKDTDFKLNELRGKKSCHTGLGRSAGWNIPMAKLYKELPDPQESIQRAAANFFSASCVPCADQSSFPKLCQLCAGKGTDKCACSNHEPYFGYSGAFKCLMEGAGDVAFVKHSTVFDNLPNPEDRKNYELLCGDNTRKSVDDYQECYLAMVPSHAVVARTVGGKEDVIWELLNHAQEHFGKDKPDNFQLFQSPHGKDLLFKDSADGFLKIPSKMDFELYLGYEYVTALQNLRESKPPDSSKDECMVKWCAIGHQERTKCDRWSGFSGGAIECETAENTEECIAKIMKGEADAMSLDGGYLYIAGKCGLVPVLAENYKTEGESCKNTPEKGYLAVAVVKTSDANINWNNLKDKKSCHTAVDRTAGWNIPMGLLYSKINNCKFDEFFSAGCAPGSPRNSSLCALCIGSEKGTGKECVPNSNERYYGYTGAFRCLVEKGDVAFVKDQTVIQNTDGNNNEAWAKNLKKENFEVLCKDGTRKPVTDAENCHLARGPNHAVVSRKDKATCVEKILNKQQDDFGKSVTDCTSNFCLFQSNSKDLLFRDDTKCLASIAKKTYDSYLGDDYVRAMTNLRQCSTSKLLEACTFHKP

SEQ ID NO: 61 - protein, 495 aa, Bos taurus [sequence omitted]

SEQ ID NO: 62 - protein, 2651 aa, Bos taurus
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNVQQKYKVSDTAATVTGYADTTIALDNSTFKASATGLGGTDQKIDGDLKFDDTTGKYYAKVTVTGGTGKDGYYEVSVDKTNGEVTLAGGATSPLTGGLPATATEDVKNVQVANADLTEAKAALTAAGVTGTASVVKMSYTDNNGKTIDGGLAVKVGDDYYSATQNKDGSISINTTKYTADDGTSKTALNKLGGADGKTEVVSIGGKTYAASKAEGHNFKAQPDLAEAAATTTENPLQKIDAALAQVDTLRSDLGAVQNRFNSAITNLGNTVNNLTSARSRIEDSDYATEVSNMSRAQILQQAGTSVLAQANQVPQNVLSLLRGGGGSGGGGSGGGGSTTTTGESADPVTGGGGSGGGGSGGGGSTGESADPVTTGGGGSGGGGSGGGGSNYGGETQTARRLHGGGGSGGGGSGGGGSETQIQRRQHTDVSFIMDRFVGGGGSGGGGSGGGGSQRRQHTDVSFIMDRFVKGGGGSGGGGSGGGGSRRQHTDVSFGGGGSGGGGSGGGGSLRTATYYFADLEVAVGGGGSGGGGSGGGGSAYHKGPFTRLGGGGSGGGGSGGGGSYSRNAVPNARGDLQVLAQKVAGGGGSGGGGSGGGGSYTVSGLSRRGDLGSLAARVAKGGGGSGGGGSGGGGSYTGGSLPNVRGDLQVLAPKAAGGGGSGGGGSGGGGSYGESDVTNVRGDLQVLAQKAAGGGGSGGGGSGGGGSYGENNVTNVRGDLQVLAQKAAGGGGSGGGGSGGGGSSKYSAPQNRRGDLGPLAARLAGGGGSGGGGSGGGGSSKYSTPQTRRGDLGPLAARLAGGGGSGGGGSGGGGSAVPNARGDLQVLAQKVARTLPGGGGSGGGGSGGGGSSLPNVRGDLQVLAPKAARPLPGGGGSGGGGSGGGGSLRGDLQVLAQKVARTLGGGGSGGGGSGGGGSRTLPTSFNYGGGGSGGGGSGGGGSTTQDRRKQEIIAPEKQTLGGGGSGGGGSGGGGSHKQKIVAPVKQTLGGGGSGGGGSGGGGSEDAVSGPNTSGGGGGSGGGGSGGGGSPFGHLTKLELPTDHHGGGGSGGGGSGGGGSYGKVSNPPRTSFPGGGGGSGGGGSGGGGSDVSLAAKHMSNTYLSGGGGSGGGGSGGGGSSIINNYYMQQYQNSMDGGGGSGGGGSGGGGSYQNSMDTQLGDNGGGGSGGGGSGGGGSPFFFSDVRSNFSKLVGGGGSGGGGSGGGGSFFRSTPEDLERAEKGGGGSGGGGSGGGGSLKARDINDIFAILKNGGGGSGGGGSGGGGSSEEKFVTMTDLVPGGGGGSGGGGSGGGGSVTMTDLVPGILEKQRGGGGSGGGGSGGGGSYFLIEKGQHEAAIEFGGGGSGGGGSGGGGSAAIEFFEGMVHDSIKGGGGSGGGGSGGGGSERTLPGQKACDDVNGGGGSGGGGSGGGGSGPYAGPLETQKPLKGGGGSGGGGSGGGGSPLERQKPLKVRAKLGGGGSGGGGSGGGGSGPYAGPMERQKPLKGGGGSGGGGSGGGGSPMERQKPLKVKAKAGGGGSGGGGSGGGGSQKPLKVKAKAPVVKGGGGSGGGGSGGGGSPVKKPVALKVKAKNGGGGSGGGGSGGGGSNADVGRLIFSGEALTGGGGSGGGGSGGGGSAVLAKDGADTFIVGTGGGGSGGGGSGGGGSMRKTKLAPTVAHGVFGGGGSGGGGSGGGGSVLDEVIFSKHKGDTKGGGGSGGGGSGGGGSTANAPLSIYEAIKGVDGLDAMEPGGGGSGGGGSGGGGSVDVLPVEHILYTRMMIGRFCGGGGSGGGGSGGGGSSATSIINTILNNIYVGGGGSGGGGSGGGGSVELDTYTMISYGDDIGGGGSGGGGSGGGGSVVASDYDLDFEALKPHFKSLGGGGSGGGGSGGGGSYDLDFEALKPHFKSLGGGGSGGGGSGGGGSEALKPHFKSLGQTYTGGGGSGGGGSGGGGSHFKSLGQTYTPADKSGGGGSGGGGSGGGGSTDVTFLKRHFHMDYGTGFYKGGGGSGGGGSGGGGSKTLEAILSFARRGTIGGGGSGGGGSGGGGSMEQLRESGPSLVKPSQTLSLTCTVSGFSLNDNRVVWVRQPPGKAPEWLGRIYSGGATHINPALKSRLSISKDNSKNEVSLSISDVRTEDTATYMCGKEQTFNDGESSYIDVWGQGFRVIVSSASTTAPKVYPLTSCCGDKSSSRVTLGCLVSSYMPEPVTVTWNSGALKSGVHTFPAVLQSSGLYSLSSMVTVPGSSSGQTFTCNVAHPASSTKVDKAVGVSSDCSKPNNQHCVREPSVFIFPPKPKDTLMITGTPEVTCVVVNVGHDNPEVQFSWFVDDVEVHTARTKPREEQFNSTYRVVSALPIQHQDWTGGKEFKCKVNIKGLSASIVRIISRSKGPAREPQVYVLDPPKEELSKSTVSLTCMVIGFYPEDVDVEWQRDRQTESEDKYRTTPPQLDADRSYFLYSKLRVDRNSWQRGDTYTCVVMHEALHNHYMQKSTSKSAGK

SEQ ID NO: 63 - protein, 2188 aa, Foot-and-mouth disease virus
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNVQQKYKVSDTAATVTGYADTTIALDNSTFKASATGLGGTDQKIDGDLKFDDTTGKYYAKVTVTGGTGKDGYYEVSVDKTNGEVTLAGGATSPLTGGLPATATEDVKNVQVANADLTEAKAALTAAGVTGTASVVKMSYTDNNGKTIDGGLAVKVGDDYYSATQNKDGSISINTTKYTADDGTSKTALNKLGGADGKTEVVSIGGKTYAASKAEGHNFKAQPDLAEAAATTTENPLQKIDAALAQVDTLRSDLGAVQNRFNSAITNLGNTVNNLTSARSRIEDSDYATEVSNMSRAQILQQAGTSVLAQANQVPQNVLSLLRTTTTGESADPVTGGGGSGGGGSGGGGSTGESADPVTTGGGGSGGGGSGGGGSNYGGETQTARRLHGGGGSGGGGSGGGGSETQIQRRQHTDVSFIMDRFVGGGGSGGGGSGGGGSQRRQHTDVSFIMDRFVKGGGGSGGGGSGGGGSRRQHTDVSFGGGGSGGGGSGGGGSLRTATYYFADLEVAVGGGGSGGGGSGGGGSAYHKGPFTRLGGGGSGGGGSGGGGSYSRNAVPNARGDLQVLAQKVAGGGGSGGGGSGGGGSYTVSGLSRRGDLGSLAARVAKGGGGSGGGGSGGGGSYTGGSLPNVRGDLQVLAPKAAGGGGSGGGGSGGGGSYGESDVTNVRGDLQVLAQKAAGGGGSGGGGSGGGGSYGENNVTNVRGDLQVLAQKAAGGGGSGGGGSGGGGSSKYSAPQNRRGDLGPLAARLAGGGGSGGGGSGGGGSSKYSTPQTRRGDLGPLAARLAGGGGSGGGGSGGGGSAVPNARGDLQVLAQKVARTLPGGGGSGGGGSGGGGSSLPNVRGDLQVLAPKAARPLPGGGGSGGGGSGGGGSLRGDLQVLAQKVARTLGGGGSGGGGSGGGGSRTLPTSFNYGGGGSGGGGSGGGGSTTQDRRKQEIIAPEKQTLGGGGSGGGGSGGGGSHKQKIVAPVKQTLGGGGSGGGGSGGGGSEDAVSGPNTSGGGGGSGGGGSGGGGSPFGHLTKLELPTDHHGGGGSGGGGSGGGGSYGKVSNPPRTSFPGGGGGSGGGGSGGGGSDVSLAAKHMSNTYLSGGGGSGGGGSGGGGSSIINNYYMQQYQNSMDGGGGSGGGGSGGGGSYQNSMDTQLGDNGGGGSGGGGSGGGGSPFFFSDVRSNFSKLVGGGGSGGGGSGGGGSFFRSTPEDLERAEKGGGGSGGGGSGGGGSLKARDINDIFAILKNGGGGSGGGGSGGGGSSEEKFVTMTDLVPGGGGGSGGGGSGGGGSVTMTDLVPGILEKQRGGGGSGGGGSGGGGSYFLIEKGQHEAAIEFGGGGSGGGGSGGGGSAAIEFFEGMVHDSIKGGGGSGGGGSGGGGSERTLPGQKACDDVNGGGGSGGGGSGGGGSGPYAGPLETQKPLKGGGGSGGGGSGGGGSPLERQKPLKVRAKLGGGGSGGGGSGGGGSGPYAGPMERQKPLKGGGGSGGGGSGGGGSPMERQKPLKVKAKAGGGGSGGGGSGGGGSQKPLKVKAKAPVVKGGGGSGGGGSGGGGSPVKKPVALKVKAKNGGGGSGGGGSGGGGSNADVGRLIFSGEALTGGGGSGGGGSGGGGSAVLAKDGADTFIVGTGGGGSGGGGSGGGGSMRKTKLAPTVAHGVFGGGGSGGGGSGGGGSVLDEVIFSKHKGDTKGGGGSGGGGSGGGGSTANAPLSIYEAIKGVDGLDAMEPGGGGSGGGGSGGGGSVDVLPVEHILYTRMMIGRFCGGGGSGGGGSGGGGSSATSIINTILNNIYVGGGGSGGGGSGGGGSVELDTYTMISYGDDIGGGGSGGGGSGGGGSVVASDYDLDFEALKPHFKSLGGGGSGGGGSGGGGSYDLDFEALKPHFKSLGGGGSGGGGSGGGGSEALKPHFKSLGQTYTGGGGSGGGGSGGGGSHFKSLGQTYTPADKSGGGGSGGGGSGGGGSTDVTFLKRHFHMDYGTGFYKGGGGSGGGGSGGGGSKTLEAILSFARRGTIGGGGSGGGGSGGGGS

SEQ ID NO: 64 - protein, 3499 aa, Foot-and-mouth disease virus
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNVQQKYKVSDTAATVTGYADTTIALDNSTFKASATGLGGTDQKIDGDLKFDDTTGKYYAKVTVTGGTGKDGYYEVSVDKTNGEVTLAGGATSPLTGGLPATATEDVKNVQVANADLTEAKAALTAAGVTGTASVVKMSYTDNNGKTIDGGLAVKVGDDYYSATQNKDGSISINTTKYTADDGTSKTALNKLGGADGKTEVVSIGGKTYAASKAEGHNFKAQPDLAEAAATTTENPLQKIDAALAQVDTLRSDLGAVQNRFNSAITNLGNTVNNLTSARSRIEDSDYATEVSNMSRAQILQQAGTSVLAQANQVPQNVLSLLRTTTTGESADPVTGGGGSGGGGSGGGGSTGESADPVTTGGGGSGGGGSGGGGSNYGGETQTARRLHGGGGSGGGGSGGGGSETQIQRRQHTDVSFIMDRFVGGGGSGGGGSGGGGSQRRQHTDVSFIMDRFVKGGGGSGGGGSGGGGSRRQHTDVSFGGGGSGGGGSGGGGSLRTATYYFADLEVAVGGGGSGGGGSGGGGSAYHKGPFTRLGGGGSGGGGSGGGGSYSRNAVPNARGDLQVLAQKVAGGGGSGGGGSGGGGSYTVSGLSRRGDLGSLAARVAKGGGGSGGGGSGGGGSYTGGSLPNVRGDLQVLAPKAAGGGGSGGGGSGGGGSYGESDVTNVRGDLQVLAQKAAGGGGSGGGGSGGGGSYGENNVTNVRGDLQVLAQKAAGGGGSGGGGSGGGGSSKYSAPQNRRGDLGPLAARLAGGGGSGGGGSGGGGSSKYSTPQTRRGDLGPLAARLAGGGGSGGGGSGGGGSAVPNARGDLQVLAQKVARTLPGGGGSGGGGSGGGGSSLPNVRGDLQVLAPKAARPLPGGGGSGGGGSGGGGSLRGDLQVLAQKVARTLGGGGSGGGGSGGGGSRTLPTSFNYGGGGSGGGGSGGGGSTTQDRRKQEIIAPEKQTLGGGGSGGGGSGGGGSHKQKIVAPVKQTLGGGGSGGGGSGGGGSEDAVSGPNTSGGGGGSGGGGSGGGGSPFGHLTKLELPTDHHGGGGSGGGGSGGGGSYGKVSNPPRTSFPGGGGGSGGGGSGGGGSDVSLAAKHMSNTYLSGGGGSGGGGSGGGGSSIINNYYMQQYQNSMDGGGGSGGGGSGGGGSYQNSMDTQLGDNGGGGSGGGGSGGGGSPFFFSDVRSNFSKLVGGGGSGGGGSGGGGSFFRSTPEDLERAEKGGGGSGGGGSGGGGSLKARDINDIFAILKNGGGGSGGGGSGGGGSSEEKFVTMTDLVPGGGGGSGGGGSGGGGSVTMTDLVPGILEKQRGGGGSGGGGSGGGGSYFLIEKGQHEAAIEFGGGGSGGGGSGGGGSAAIEFFEGMVHDSIKGGGGSGGGGSGGGGSERTLPGQKACDDVNGGGGSGGGGSGGGGSGPYAGPLETQKPLKGGGGSGGGGSGGGGSPLERQKPLKVRAKLGGGGSGGGGSGGGGSGPYAGPMERQKPLKGGGGSGGGGSGGGGSPMERQKPLKVKAKAGGGGSGGGGSGGGGSQKPLKVKAKAPVVKGGGGSGGGGSGGGGSPVKKPVALKVKAKNGGGGSGGGGSGGGGSNADVGRLIFSGEALTGGGGSGGGGSGGGGSAVLAKDGADTFIVGTGGGGSGGGGSGGGGSMRKTKLAPTVAHGVFGGGGSGGGGSGGGGSVLDEVIFSKHKGDTKGGGGSGGGGSGGGGSTANAPLSIYEAIKGVDGLDAMEPGGGGSGGGGSGGGGSVDVLPVEHILYTRMMIGRFCGGGGSGGGGSGGGGSSATSIINTILNNIYVGGGGSGGGGSGGGGSVELDTYTMISYGDDIGGGGSGGGGSGGGGSVVASDYDLDFEALKPHFKSLGGGGSGGGGSGGGGSYDLDFEALKPHFKSLGGGGSGGGGSGGGGSEALKPHFKSLGQTYTGGGGSGGGGSGGGGSHFKSLGQTYTPADKSGGGGSGGGGSGGGGSTDVTFLKRHFHMDYGTGFYKGGGGSGGGGSGGGGSKTLEAILSFARRGTIGGGGSGGGGSGGGGSMKWVTFISLLLLFSSAYSRGVFRRDTHKSEIAHRFKDLGEEHFKGLVLIAFSQYLQQCPFDEHVKLVNELTEFAKTCVADESHAGCEKSLHTLFGDELCKVASLRETYGDMADCCEKQEPERNECFLSHKDDSPDLPKLKPDPNTLCDEFKADEKKFWGKYLYEIARRHPYFYAPELLYYANKYNGVFQECCQAEDKGACLLPKIETMREKVLASSARQRLRCASIQKFGERALKAWSVARLSQKFPKAEFVEVTKLVTDLTKVHKECCHGDLLECADDRADLAKYICDNQDTISSKLKECCDKPLLEKSHCIAEVEKDAIPENLPPLTADFAEDKDVCKNYQEAKDAFLGSFLYEYSRRHPEYAVSVLLRLAKEYEATLEECCAKDDPHACYSTVFDKLKHLVDEPQNLIKQNCDQFEKLGEYGFQNALIVRYTRKVPQVSTPTLVEVSRSLGKVGTRCCTKPESERMPCTEDYLSLILNRLCVLHEKTPVSEKVTKCCTESLVNRRPCFSALTPDETYVPKAFDEKLFTFHADICTLPDTEKQIKKQTALVELLKHKPKATEEQLKTVMENFVAFVDKCCAADDKEACFAVEGPKLVVSTQTALAMRPAVRALLACAVLGLCLADPERTVRWCTISTHEANKCASFRENVLRILESGPFVSCVKKTSHMDCIKAISNNEADAVTLDGGLVYEAGLKPNNLKPVVAEFHGTKDNPQTHYYAVAVVKKDTDFKLNELRGKKSCHTGLGRSAGWNIPMAKLYKELPDPQESIQRAAANFFSASCVPCADQSSFPKLCQLCAGKGTDKCACSNHEPYFGYSGAFKCLMEGAGDVAFVKHSTVFDNLPNPEDRKNYELLCGDNTRKSVDDYQECYLAMVPSHAVVARTVGGKEDVIWELLNHAQEHFGKDKPDNFQLFQSPHGKDLLFKDSADGFLKIPSKMDFELYLGYEYVTALQNLRESKPPDSSKDECMVKWCAIGHQERTKCDRWSGFSGGAIECETAENTEECIAKIMKGEADAMSLDGGYLYIAGKCGLVPVLAENYKTEGESCKNTPEKGYLAVAVVKTSDANINWNNLKDKKSCHTAVDRTAGWNIPMGLLYSKINNCKFDEFFSAGCAPGSPRNSSLCALCIGSEKGTGKECVPNSNERYYGYTGAFRCLVEKGDVAFVKDQTVIQNTDGNNNEAWAKNLKKENFEVLCKDGTRKPVTDAENCHLARGPNHAVVSRKDKATCVEKILNKQQDDFGKSVTDCTSNFCLFQSNSKDLLFRDDTKCLASIAKKTYDSYLGDDYVRAMTNLRQCSTSKLLEACTFHKP

SEQ ID NO: 65 - protein, 21 aa, synthetic construct
DDDDKNQTPGNARILQTMKGL

SEQ ID NO: 66 - protein, 17 aa, synthetic construct
KQNQQSSSEQDYSKKKK

SEQ ID NO: 67 - protein, 11 aa, synthetic construct
DICLPRWGCLW

SEQ ID NO: 68 - protein, 4 aa, synthetic construct
DAHK

SEQ ID NO: 69 - protein, 6 aa, synthetic construct
HWRGWV

SEQ ID NO: 70 - protein, 1753 aa, Foot-and-mouth disease virus
DICLPRWGCLWGGGGSGGGGSGGGGSTHRPPMWSPVWPGGGGSGGGGSGGGGSHWRGWVGGGGSGGGGSGGGGSTTTTGESADPVTGGGGSGGGGSGGGGSTGESADPVTTGGGGSGGGGSGGGGSNYGGETQTARRLHGGGGSGGGGSGGGGSETQIQRRQHTDVSFIMDRFVGGGGSGGGGSGGGGSQRRQHTDVSFIMDRFVKGGGGSGGGGSGGGGSRRQHTDVSFGGGGSGGGGSGGGGSLRTATYYFADLEVAVGGGGSGGGGSGGGGSAYHKGPFTRLGGGGSGGGGSGGGGSYSRNAVPNARGDLQVLAQKVAGGGGSGGGGSGGGGSYTVSGLSRRGDLGSLAARVAKGGGGSGGGGSGGGGSYTGGSLPNVRGDLQVLAPKAAGGGGSGGGGSGGGGSYGESDVTNVRGDLQVLAQKAAGGGGSGGGGSGGGGSYGENNVTNVRGDLQVLAQKAAGGGGSGGGGSGGGGSSKYSAPQNRRGDLGPLAARLAGGGGSGGGGSGGGGSSKYSTPQTRRGDLGPLAARLAGGGGSGGGGSGGGGSAVPNARGDLQVLAQKVARTLPGGGGSGGGGSGGGGSSLPNVRGDLQVLAPKAARPLPGGGGSGGGGSGGGGSLRGDLQVLAQKVARTLGGGGSGGGGSGGGGSRTLPTSFNYGGGGSGGGGSGGGGSTTQDRRKQEIIAPEKQTLGGGGSGGGGSGGGGSHKQKIVAPVKQTLGGGGSGGGGSGGGGSEDAVSGPNTSGGGGGSGGGGSGGGGSPFGHLTKLELPTDHHGGGGSGGGGSGGGGSYGKVSNPPRTSFPGGGGGSGGGGSGGGGSDVSLAAKHMSNTYLSGGGGSGGGGSGGGGSSIINNYYMQQYQNSMDGGGGSGGGGSGGGGSYQNSMDTQLGDNGGGGSGGGGSGGGGSPFFFSDVRSNFSKLVGGGGSGGGGSGGGGSFFRSTPEDLERAEKGGGGSGGGGSGGGGSLKARDINDIFAILKNGGGGSGGGGSGGGGSSEEKFVTMTDLVPGGGGGSGGGGSGGGGSVTMTDLVPGILEKQRGGGGSGGGGSGGGGSYFLIEKGQHEAAIEFGGGGSGGGGSGGGGSAAIEFFEGMVHDSIKGGGGSGGGGSGGGGSERTLPGQKACDDVNGGGGSGGGGSGGGGSGPYAGPLETQKPLKGGGGSGGGGSGGGGSPLERQKPLKVRAKLGGGGSGGGGSGGGGSGPYAGPMERQKPLKGGGGSGGGGSGGGGSPMERQKPLKVKAKAGGGGSGGGGSGGGGSQKPLKVKAKAPVVKGGGGSGGGGSGGGGSPVKKPVALKVKAKNGGGGSGGGGSGGGGSNADVGRLIFSGEALTGGGGSGGGGSGGGGSAVLAKDGADTFIVGTGGGGSGGGGSGGGGSMRKTKLAPTVAHGVFGGGGSGGGGSGGGGSVLDEVIFSKHKGDTKGGGGSGGGGSGGGGSTANAPLSIYEAIKGVDGLDAMEPGGGGSGGGGSGGGGSVDVLPVEHILYTRMMIGRFCGGGGSGGGGSGGGGSSATSIINTILNNIYVGGGGSGGGGSGGGGSVELDTYTMISYGDDIGGGGSGGGGSGGGGSVVASDYDLDFEALKPHFKSLGGGGSGGGGSGGGGSYDLDFEALKPHFKSLGGGGSGGGGSGGGGSEALKPHFKSLGQTYTGGGGSGGGGSGGGGSHFKSLGQTYTPADKSGGGGSGGGGSGGGGSTDVTFLKRHFHMDYGTGFYKGGGGSGGGGSGGGGSKTLEAILSFARRGTIG

SEQ ID NO: 71 - protein, 12 aa, synthetic construct
THRPPMWSPVWP